SEQUENCE LISTING (43 sequences)

SEQ ID NO: 1 - protein, 334 aa, Mus musculus
MNLLLLLAVLCLGTALATPKFDQTFSAEWHQWKSTHRRLYGTNEEEWRRAIWEKNMRIIQLHNGEYSNGQHGFSMEMNAFGDMTNEEFRQVVNGYRHQKHKKGRLFQEPLMLKIPKSVDWREKGCVTPVKNQGQCGSCWAFSASGCLEGQMFLKTGKLISLSEQNLVDCSHAQGNQGCNGGLMDFAFQYIKENGGLDSEESYPYEAKDGSCKYRAEFAVANDTGFVDIPQQEKALMKAVATVGPISVAMDASHPSLQFYSSGIYYEPNCSSKNLDHGVLLVGYGYEGTDSNKNKYWLVKNSWGSEWGMEGYIKIAKDRDNHCGLATAASYPVVN

SEQ ID NO: 2 - protein, 333 aa, Homo sapiens [sequence omitted]

SEQ ID NO: 3 - protein, 19 aa, Mus musculus
NQGCNGGLMDFAFQYIKEN

SEQ ID NO: 4 - protein, 19 aa, Homo sapiens
NEGCNGGLMDYAFQYVQDN

SEQ ID NO: 5 - protein, 21 aa, Mus musculus
GGLDSEESYPYEAKDGSCKYR

SEQ ID NO: 6 - protein, 21 aa, Homo sapiens
GGLDSEESYPYEATEESCKYN

SEQ ID NO: 7 - DNA, 423 bp, Mus musculus
  source 1..423: mol_type = other DNA
atgaaatgcagctgggttatcttcttcctgatgacagtggttacaggggtcaattcagagggtcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgattgttaatactagatatgacccgaagttccaggacaaggccactataacagcagacacatcctccaacacagcctacctgcagctcagcagcctgacatctgaggacactgccgtctattactgtgctagattgggtaattactacggtagtaggtatgttatggactactggggtcaaggaacctcagtcaccgtctcctca

SEQ ID NO: 8 - protein, 141 aa, Mus musculus
MKCSWVIFFLMTVVTGVNSEGQLQQSGAELVKPGASVKLSCTASGFNIKDTYMHWVKQRPEQGLEWIGRIDPAIVNTRYDPKFQDKATITADTSSNTAYLQLSSLTSEDTAVYYCARLGNYYGSRYVMDYWGQGTSVTVSS

SEQ ID NO: 9 - DNA, 393 bp, Mus musculus
  source 1..393: mol_type = other DNA
atgaagttgcctgctaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 10 - protein, 131 aa, Mus musculus
MKLPARLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 11 - DNA, 408 bp, Mus musculus
  source 1..408: mol_type = other DNA
atgaaattcagctgggtcatcttcttcctgatggcagtggttacaggggtcaattcagaggttcagctgcagcagtctgtggcagagcttgtgaggccaggggcctcagtcaagttgtcctgcacagcttctggcctcaatattaaaaacacctatatgcactgggtgaagcagaggcctgaacagggcctggagtggattggaaggattgatcctacgaatggtaatactaaatatgccccgaagttccagggcaaggccactataactgcagacacatcctccagcacagcctacctgcagctcagcagcctgacatctgaggacactgccatctattactgtgctaagggggattacggcgacggggcttactggggccaagggactctggtctctgtctctgca

SEQ ID NO: 12 - protein, 136 aa, Mus musculus
MKFSWVIFFLMAVVTGVNSEVQLQQSVAELVRPGASVKLSCTASGLNIKNTYMHWVKQRPEQGLEWIGRIDPTNGNTKYAPKFQGKATITADTSSSTAYLQLSSLTSEDTAIYYCAKGDYGDGAYWGQGTLVSVSA

SEQ ID NO: 13 - DNA, 393 bp, Mus musculus
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 14 - protein, 131 aa, Mus musculus
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 15 - DNA, 417 bp, Mus musculus
  source 1..417: mol_type = other DNA
atgaaattcagctgggtcatcttcttcctgatggcagtggttacaggggtcaattcagaggttcagctgcagcagtctgtggcagagcttgtgaggccaggggcctcagtcaagttgtcctgcacaacttctggcttcaacattaaaaacacctatatgtattgggtgaagcagaggcctgaacagggcctggagtggattggaaggattgatcctgcgaatggtaatattaaatatgacccgaagttccagggcaaggccactataattgcagacacatcctccaacacagcctacctgcagttcagaagcctgacatctgaggacaatgccatctattactgtgctagtcggaaggtaatgactgcggactactttgactactggggccaaggcaccactctcacagtctcctca

SEQ ID NO: 16 - protein, 139 aa, Mus musculus
MKFSWVIFFLMAVVTGVNSEVQLQQSVAELVRPGASVKLSCTTSGFNIKNTYMYWVKQRPEQGLEWIGRIDPANGNIKYDPKFQGKATIIADTSSNTAYLQFRSLTSEDNAIYYCASRKVMTADYFDYWGQGTTLTVSS

SEQ ID NO: 17 - DNA, 393 bp, Mus musculus
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 18 - protein, 131 aa, Mus musculus
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 19 - protein, 20 aa, Mus musculus
NLDHGVLLVGYGYEGTDSNK

SEQ ID NO: 20 - protein, 14 aa, Mus musculus
NSWGSEWGMEGYIK

SEQ ID NO: 21 - protein, 16 aa, Mus musculus
ENGGLDSEESYPYEAK

SEQ ID NO: 22 - protein, 14 aa, Mus musculus
NSWGSEWGMEGYIK

SEQ ID NO: 23 - protein, 10 aa, Mus musculus
LYGTNEEEWR

SEQ ID NO: 24 - protein, 13 aa, Mus musculus
FDQTFSAEWHQWK

SEQ ID NO: 25 - protein, 11 aa, Mus musculus
GRLFQEPLMLK

SEQ ID NO: 26 - protein, 12 aa, Mus musculus
RLYGTNEEEWRR

SEQ ID NO: 27 - protein, 9 aa, Mus musculus
LFQEPLMLK

SEQ ID NO: 28 - DNA, 18 bp, Mus musculus
  source 1..18: mol_type = other DNA
ggaggagagcgatatggg

SEQ ID NO: 29 - DNA, 18 bp, Mus musculus
  source 1..18: mol_type = other DNA
agccattcaccacctgcc

SEQ ID NO: 30 - DNA, 20 bp, Mus musculus
  source 1..20: mol_type = other DNA
aattcgccaatgacaagacg

SEQ ID NO: 31 - protein, 5 aa, Mus musculus
DTYMH

SEQ ID NO: 32 - protein, 17 aa, Mus musculus
RIDPAIVNTRYDPKFQD

SEQ ID NO: 33 - protein, 13 aa, Mus musculus
LGNYYGSRYVMDY

SEQ ID NO: 34 - protein, 16 aa, Mus musculus
RSSQSLVHSNGNTYLH

SEQ ID NO: 35 - protein, 7 aa, Mus musculus
KVSNRFS

SEQ ID NO: 36 - protein, 9 aa, Mus musculus
SQSTHVPYT

SEQ ID NO: 37 - protein, 5 aa, Mus musculus
NTYMH

SEQ ID NO: 38 - protein, 17 aa, Mus musculus
RIDPTNGNTKYAPKFQG

SEQ ID NO: 39 - protein, 8 aa, Mus musculus
GDYGDGAY

SEQ ID NO: 40 - protein, 5 aa, Mus musculus
NTYMY

SEQ ID NO: 41 - protein, 17 aa, Mus musculus
RIDPANGNIKYDPKFQG

SEQ ID NO: 42 - protein, 11 aa, Mus musculus
RKVMTADYFDY

SEQ ID NO: 43 - protein, 9 aa, Mus musculus
SQSTHVPWT

(19) United States
(12) Patent Application Publication
(10) Pub. No.: US 2026/0347504 A1
(43) Pub. Date: Nov. 5, 2026

(54) USE OF PROCATHEPSIN L-NEUTRALIZING MONOCLONAL ANTIBODIES TO TREAT SEPSIS, RHEUMATOID ARTHRITIS, AND OTHER INFLAMMATORY DISEASES

(71) Applicant: THE FEINSTEIN INSTITUTES FOR MEDICAL RESEARCH, Manhasset, NY (US)

(72) Inventors: Haichao WANG, Edison, NJ (US); Cassie ZHU, Great Neck, NY (US)

(73) Assignee: THE FEINSTEIN INSTITUTES FOR MEDICAL RESEARCH, Manhasset, NY (US)

(21) Appl. No.: 19/119,886

(22) PCT Filed: Oct. 5, 2023
(86) PCT No.: PCT/US2023/034513

§ 371 Date: Apr. 10, 2025

Related U.S. Application Data
(60) Provisional application No. 63/415,708, filed on Oct. 13, 2022.

(51) Int. Cl. C07K 16/40; A61P 37/06

(52) U.S. Cl. CPC C07K 16/40; A61P 37/06

(57) ABSTRACT

Provided are monoclonal antibodies that specifically bind to human precathepsin L, antigen-binding fragments thereof, and pharmaceutical compositions comprising the antibodies and fragments, as well as methods of treatment of sepsis, rheumatoid arthritis and other inflammatory diseases using the antibodies and fragments. Also provided are nucleic acids encoding the antibodies and fragments, expression vectors comprising the nucleic acid, and host cells transformed with the expression vectors.

### CROSS-REFERENCE TO RELATED APPLICATION

This application claims the benefit of U.S. Provisional Patent Application No. 63/415,708, filed on Oct. 13, 2022, the contents of which are herein incorporated by reference in their entirety into the present application.

### STATEMENT OF GOVERNMENT SUPPORT

This invention was made with government support under grant numbers AT005076, GM063075 and GM145331 awarded by the National Institutes of Health. The government has certain rights in the invention.

### INCORPORATION BY REFERENCE OF SEQUENCE LISTING XML

The Sequence Listing entitled “813.xml”, created on Sep. 29, 2023, 41 KB, submitted electronically using the U.S. Patent Center is incorporated by reference as the Sequence Listing XML for the subject application.

### BACKGROUND

Throughout this application various publications are referred to in parentheses. Full citations for these may be found at the end of the specification immediately preceding the claims. The disclosures of these publications are hereby incorporated by reference in their entireties into the subject application to more fully describe the art to which the subject application pertains. The discussion of these publications herein is intended merely to summarize the assertions made by Applicant and no admission is made that any publication constitutes prior art.

Microbial infections and resultant sepsis syndromes are the most common causes of death in intensive care units, accounting for approximately 20% of total deaths worldwide (1). The pathogenesis of sepsis remains poorly understood, but is partly attributable to dysregulated innate immune responses (e.g., hyperinflammation and immunosuppression) to lethal infections (2). To mount efficient inflammatory responses, innate immune cells employ various pattern recognition receptors [PRRs. e.g., the toll-like receptor 4 (TLR4)](3) to recognize distinct classes of molecules shared by related microbes known as “pathogen-associated molecular patterns” [PAMPs. e.g., bacterial endotoxins, lipopolysaccharide (LPS)]. For instance, upon detecting minute amount of endotoxins by an LPS-binding protein (LBP) (4), a co-receptor cluster of differentiation 14 (CD14) (5) delivers it to the high-affinity cell-surface PRR, TLR4 (3), thereby triggering the immediate release of “early” cytokines such as tumor necrosis factor (TNF) (6), interleukin-1β (IL-1β) (7) and interferon-γ (IFN-γ) (8).

However, if excessive amounts of LPS is internalized via CD14/TLR4-mediated endocytosis or bacterial outer membrane vesicles (9), it induces oligomerization and activation of cytoplasmic Casp-11/4/5 receptor (10), resulting in dysregulated pyroptosis and consequent leakage of late-acting damage-associated molecular patterns (DAMPs) such as high mobility group box 1 (HMGB1) (11) and sequestosome-1 (SQSTM1) (12). When HMGB1 was secreted by innate immune cells in relatively low amounts, it binds high-affinity TLR4 receptor to augment inflammation during an early stage of sepsis (13). However, when HMGB1 is passively released by pyroptotic cells in overwhelmingly higher quantities, it could also bind to other low affinity receptors, such as the receptor for advanced glycation end product (RAGE), thereby inducing immune tolerance (14), pyroptosis (15, 16) and immunosuppression (17) that may adversely compromise the host's ability to eradicate microbial infections (18). Consequently, HMGB1 has been characterized as a late-acting DAMP and mediator of lethal sepsis with a relatively wider therapeutic window than early proinflammatory cytokines (11, 13, 19).

In parallel, early cytokines (e.g., TNF, IL-1β and IFN-γ) also induce other “intermediate” pro-inflammatory mediators such as serum amyloid A (SAA) in hepatocytes (20). Upon secretion, extracellular SAA employs TLR4 (21) and RAGE (22) to induce hemichannels (e.g., connexin 43 and pannexin 1) (23, 24) and secretory phospholipase A₂ (e.g., sPLA₂-IIE/V) (25), thereby triggering HMGB1 release and serving as a mediator of lethal sepsis (26). However, it was previously unknown whether SAA also induces other “late-acting” cytokines that can be therapeutically targeted in a delayed regimen.

Cathepsin L (CTS-L) is a papain-like lysosomal enzyme responsible for degrading endocytosed proteins to generate immunogenic antigens for adaptive immunities. Although human and murine pCTS-L share nearly 86% sequence homology, they exhibit only 59% homology to the counterpart pCTS-L of a distantly related liver fluke parasite (27). Unlike other papain enzymes, CTS-L is inducible in some malignantly transformed tumor cells by various growth factors, and the leader-less precursor, proCTS-L (pCTS-L), can be secreted extracellularly (28) to facilitate tumor invasion and metastasis (29). Even in non-transformed cells, some inflammatory (e.g., LPS, IFN-γ or IL-6) (30, 31) and noxious stimuli (e.g., alcohol consumption, cigarette smoking, and UV irradiation) (32-34) similarly stimulate the expression and secretion of pCTS-L in innate immune cells (30, 33) or non-immune cells such as hepatocytes (32), dermal fibroblasts (34) and synovial fibroblasts (31). However, it was previously unknown i) whether an intermediate mediator, SAA, similarly induces pCTS-L expression and secretion; and ii) whether pCTS-L can be therapeutically targeted in a delayed fashion.

Inflammatory arthritis, such as the rheumatoid arthritis (RA), is characterized by synovial inflammation that often leads to joint pain, functional limitation, and progressive and irreversible damage to the joints (35). In the U.S. alone, RA affects approximately 1.5 million individuals, escalating as an important cause of disability. The development of biologics targeting specific molecular targets is urgently needed for pharmacologic treatment of various rheumatic diseases (36). For instance, monoclonal antibodies against tumor necrosis factor (TNF) were first developed as a biological medication for patients with RA (36). A few decades after the development of the 1^{st} anti-TNF drug (etanercept), more than 10 different biological agents have been approved by the U.S. Food and Drug Administration (FDA). Given the lack of effective therapies, it is still important to develop novel therapeutics for the clinical management of human arthritis and other inflammatory diseases.

Recently, we discovered that the nascent precursor of a lysosomal enzyme, procathepsin L (pCTS-L), can be secreted by activated innate immune cells, and contributes to dysregulated inflammation in experimental sepsis (37). On one hand, pCTS-L binds various pattern recognition receptors such as the toll-like receptor 4 (TLR4) and the receptor for advanced glycation end products (RAGE), and induces the production of various cytokines (e.g., IL-6 and TNF) and chemokines (e.g., RANTES, MCP-1, ENA-78/LIX, IL-8, GRO-α/KC, and GRO-α/β/γ). On the other hand, CTS-L may also contribute to the degradation of cartilage matrix component during the progression of chronic RA (38, 39). Clinically, the levels of CTS-L in both the serum and synovial fluid of RA patients were markedly elevated (40), supporting a possible role of pCTS-L in the pathogenesis of arthritis. In an experimental model of arthritis, genetic deletion of Ctsl led to a significant attenuation of disease severity as manifested by a significant reduction in joint swelling, inflammation, and destruction (41).

The present invention provides for a novel role for extracellular pCTS-L in the pathogenesis of lethal sepsis; and a therapeutic use of a panel of pCTS-L-neutralizing monoclonal antibodies (mAbs) in the treatment of sepsis, rheumatoid arthritis, and other inflammatory diseases.

### SUMMARY OF THE INVENTION

The invention provides a monoclonal antibody specifically binding to amino acid sequence GGLDSEESYPYEATEESCKYN (SEQ ID NO: 6) present in residues 194-214 of human precathepsin L (SEQ ID NO: 2), or an antigen-binding fragment thereof, comprising a heavy chain variable region (VH) and a light chain variable region (VL), wherein
(A)
   (i) the heavy chain variable region (VH) comprises:
   (a) an amino acid sequence of heavy chain CDR1 comprising the amino acid sequence DTYMH (SEQ ID NO: 31);
   (b) an amino acid sequence of heavy chain CDR2 comprising the amino acid sequence RIDPAIVNTRYDPKFQD (SEQ ID NO: 32); and
   (c) an amino acid sequence of heavy chain CDR3 comprising the amino acid sequence LGNYYGSRYVMDY (SEQ ID NO: 33); and
   (ii) the light chain variable region (VL) comprises:
   (a) an amino acid sequence of light chain CDR1 comprising the amino acid sequence RSSQSLVHSNGNTYLH (SEQ ID NO: 34);
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPYT (SEQ ID NO: 36):
   or
(B)
   (i) the heavy chain variable region (VH) comprises:
   (a) an amino acid sequence of heavy chain CDR1 comprising the amino acid sequence NTYMH (SEQ ID NO: 37);
   (b) an amino acid sequence of heavy chain CDR2 comprising the amino acid sequence RIDPTNGNTKYAPKFQG (SEQ ID NO: 38); and
   (c) an amino acid sequence of heavy chain CDR3 comprising the amino acid sequence GDYGDGAY (SEQ ID NO: 39); and
   (ii) the light chain variable region (VL) comprises:
   (a) an amino acid sequence of light chain CDR1 comprising the amino acid sequence RSSQSLVHSNGNTYLH (SEQ ID NO: 34);
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPYT (SEQ ID NO: 36);
   or
(C)
   (i) the heavy chain variable region (VH) comprises:
   (a) an amino acid sequence of heavy chain CDR1 comprising the amino acid sequence NTYMY (SEQ ID NO: 40);
   (b) an amino acid sequence of heavy chain CDR2 comprising the amino acid sequence RIDPANGNIKYDPKFQG (SEQ ID NO: 41); and
   (c) an amino acid sequence of heavy chain CDR3 comprising the amino acid sequence RKVMTADYFDY (SEQ ID NO: 42); and
   (ii) the light chain variable region (VL) comprises:
   (a) an amino acid sequence of light chain CDR1 comprising the amino acid sequence RSSQSLVHSNGNTYLH (SEQ ID NO: 34);
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPWT (SEQ ID NO: 43).

Also provided are pharmaceutical compositions comprising the antibodies and fragments, as well as methods of treatment of sepsis, rheumatoid arthritis and other inflammatory diseases using the antibodies and fragments. Further provided are nucleic acids encoding the antibodies and fragments, expression vectors comprising the nucleic acid, and host cells transformed with the expression vectors.

These and other embodiments are disclosed or are obvious from and encompassed by the following Detailed Description of the Invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A-1B. Identification of procathepsin L (pCTS-L) as an inducible and secretory protein from activated innate immune cells.

A) Characterization of pCTS-L as a secretory protein in SAA-stimulated murine macrophages (MΦ). Murine macrophage-like RAW 264.7 cells were stimulated with SAA for 16 h, and extracellular proteins in the macrophage-conditioned culture medium were resolved by SDS-PAGE gel electrophoresis. A 40-kDa SAA-inducible secretory protein (“P40”) was identified as pCTS-L by mass spectrometrv. Peptide SEQ ID NOs. from top to bottom of panel: SEQ ID NO: 19-SEQ ID NO: 27, respectively.

B) Induction and secretion of pCTS-L in LPS- and SAA-stimulated human PBMCs. Human PBMCs were stimulated with LPS, HMGB1 or SAA for 16 h, and levels of pCTS-L in the cell-conditioned medium (“pCTS-L”) were determined by Western blotting analysis. Sample loading was normalized by equal volume of culture medium conditioned by equivalent number of cells. Bar graph represents quantitation of pCTS-L band intensities of six independent experiments in arbitrary unit (AU). *, P<0.05 VS “-LPS-SAA-HMGB1”, one way ANOVA test.

FIG. 2A-2B. Blood pCTS-L protein concentrations were time-dependently elevated in experimental sepsis.

A) Upregulation of Ctsl mRNA in experimental sepsis. Male and female Balb/C mice were sacrificed at 24 h post CLP to harvest various tissues to isolate total mRNAs for real-time RT-PCR analysis of Ctsl mRNA expression with reference to a house-keeping gene, glyceraldehyde 3-phosphate dehydrogenase (GAPDH). *, P<0.05 versus “-CLP”, one-way ANOVA.

B) Blood pCTS-L protein levels were time-dependently increased in experimental sepsis. Male Balb/C mice were sacrificed at various time points after CLP surgery to harvest blood samples for measuring serum pCTS-L contents by Western blotting with reference to highly purified recombinant pCTS-L at various dilutions. N=5 mice per time point. *, P<0.05 versus “—CLP”, one-way ANOVA.

FIG. 3A-3C. Blood pCTS-L levels were elevated in septic patients, and positively correlated with surrogate markers of experimental and clinical sepsis.

A) Western blotting of plasma pCTS-L in a representative normal healthy control (“H”) and a septic patient (“S”) at three time points after diagnosis.

B) Cytokine Antibody Array analysis of 42 cytokines and chemokines in a representative normal healthy control (“H”) and a septic patient at three time points post the clinical diagnosis.

C) Correlation between plasma pCTS-L level and SOFA score as well as surrogate markers of sepsis. The Spearman rank correlation coefficient test was used to evaluate associations between two variables that exhibit non-normal distribution.

FIG. 4A-4B. Expression and purification of recombinant human and murine procathepsin L (pCTS-L).

A) Amino acid sequence and structural domains of murine and human precathepsin L (preCTS-L; SEQ ID NO: 1 and SEQ ID NO: 2 respectively). Human and murine pre-cathepsin L (pre-CTS-L) are synthesized as a 333 or 334 amino acid precursor with an N-terminal 16- or 17-residue leader signal sequence, which can be removed in the ER to release the procathepsin L (pCTS-L). The pCTS-L is folded with the assistance of three disulfide bonds (not shown) and the pro-region, which can then be cleaved in the endosome to release the cathepsin L (CTS-L). Upon delivery to the lysosome, the CTS-L is further proteolytically processed to produce the active enzyme consisting of a heavy and light chain.

B) Expression and purification of recombinant human and murine pCTS-L proteins. Recombinant human (“H”) and murine (“M”) pCTS-L corresponding to residue 17-333 or 18-334 of respective precathepsin L with a N-histidine tag were expressed in E. coli BL21 (DE3) pLysS cells as insoluble inclusion bodies. After sonication to disrupt the bacteria, the inclusion bodies were isolated by differential centrifugation following extensive washing in 1×PBS containing 1% Triton X-100. The inclusion bodies were then solubilized in 8 M urea, and refolded by dialysis in 10 mM Tris buffer (pH 8.0) containing N-lauroylsarcosine. Subsequently, the recombinant proteins were subjected to extensive Triton X-114 extractions to remove contaminating endotoxins.

FIG. 5. pCTS-L dose-dependently interacted with TLR4 and RAGE receptor.

Highly purified recombinant pCTS-L or recombinant protein corresponding to entire human TLR4 (residue 1-631) or the extracellular domain of RAGE (residue 1-344) was immobilized on the NTA sensor chip, and various analytes (e.g., TLR4, RAGE, or pCTS-L) were respectively applied as analyte at different concentrations. The response units were recorded over time, and the binding affinity was estimated as the equilibrium dissociation constant K_{D}. Shown were representative K_{D} or mean±SEM of eight independent experiments.

FIG. 6A-6B. Intraperitoneal administration of recombinant pCTS-L induced hepatic fibrinogen expression in mice.

A) Heat map of top 30 genes differentially expressed in the pCTS-L-challenged Balb/C mice. A bi-clustering heat map was used to visualize the expression profile of the top 30 differentially expressed genes that were sorted by their adjusted P-value and log 2 fold of changes. Genes with an adjusted P-value<0.05 and absolute log 2 fold change>2 were defined as differentially expressed. Each row represents a gene and each column represents one sample from each animal.

B) pCTS-L markedly elevated hepatic fibrinogen-γ (FGG) protein content. Recombinant pCTS-L was intraperitoneally administered into Balb/C mice at a pathological dose (20 mg/kg), and hepatic tissues were harvested 20 h later to measure hepatic FGG content by Western botting. *P<0.05 vs. normal control (“-pCTS-L”).

FIG. 7. Disruption of Ctsl expression attenuated sepsis-induced liver injury.

Wildtype or Ctsl KO mice were subjected to CLP surgery, and liver was harvested at 24 h post CLP for H&E staining and histological analysis. Liver injury scores were expressed as means f SEM of 3-4 animals per group. *P<0.05 vs. negative control (“-CLP”); #, P<0.05 vs. positive control (“+CLP”) of WT.

FIG. 8A-8B. Sequence of 24 synthetic peptides of human and murine pCTS-L and epitope mapping of anti-pCTS-L rabbit and murine serum.

A) Sequence of 24 peptides corresponding to indicated regions of murine and human pCTS-L (SEQ ID NOs: 1 and 2, respectively). The sequences for Peptide 12 (P12) and Peptide 13 (P13) of murine and human pCTS-L protein were designated as SEQ ID NOs: 3, 4, 5 and 6, respectively.

B) Epitope mapping of rabbit or murine serum raised against murine or human pCTS-L.

FIG. 9A-9B. Anti-pCTS-L polyclonal IgGs (pAbs) significantly attenuated pCTS-L-induced inflammation.

A) Scheme for purifying pCTS-L antigen-binding IgGs from rabbit total IgGs. Total IgGs were purified from anti-murine pCTS-L rabbit serum using Protein A affinity chromatography, and pCTS-L-binding IgGs were then purified by pCTS-L antigen-affinity chromatography. The non-pCTS-L-binding control immunoglobulins (“C-IgG”) were collected in the washout fractions before the pCTS-L antigen-bound antibodies (“A-IgGs”) were eluted from the column by an acidic buffer into solution with physiological pH to prevent acid-catalyzed antibody denaturation.

B) pCTS-L antigen-affinity purified IgGs abrogated the pCTS-L-induced cytokines and chemokines. Thioglycollate-elicited peritoneal macrophages were isolated from Balb/C mice, and stimulated with recombinant pCTS-L either alone or in the presence of control IgGs (C-IgGs) or antigen-affinity purified IgGs (“A-IgGs”) for 16 h, and the extracellular levels of 62 different cytokines and chemokines were measured by Cytokine Antibody Arrays and expressed as mean±SEM of two experiments in duplicates (n=4) in arbitrary units. *, P<0.05 versus “-pCTS-L” negative control; #, P<0.05 versus “+pCTS-L” positive control. Note that antigen-affinity purified IgGs effectively abrogated the pCTS-L-induced secretion of IL-6, sTNF-RII and six different chemokines such as RANTES, MCP-1, MIP-1γ, LIX. MIP-2 and KC.

FIG. 10A-10B. pCTS-L-specific polyclonal and monoclonal antibodies (mAbs) conferred a significant protection against lethal sepsis.

A) Conformation of human pCTS-L and two mAb-targeting peptides. The position of two epitope peptides (P12 and P13) was marked, and their sequences for murine and human pCTS-L protein were designated as SEQ ID NOs: 3, 4, 5 and 6, respectively.

B) Polyclonal and monoclonal antibodies (pAbs and mAbs) raised against murine or human pCTS-L rescued mice from lethal sepsis. Male or female Balb/C mice were subjected to CLP, and polyclonal or monoclonal antibodies (pAbs or mAbs) against two different epitopes (P12 or P13) of human and murine pCTS-L were given intraperitoneally at indicated doses and time points, and animal survival rates were monitored for two weeks to ensure no later death. *, P<0.05 versus saline control group.

FIG. 11A-11B. Epitope mapping and antigen affinities of human and murine pCTS-L-reactive monoclonal antibodies.

A) Use of eight pairs of homologous peptides corresponding to different regions of murine (M) or human (H) pCTS-L to determine the epitope profile of monoclonal antibodies raised against murine pCTS-L. The isotype, relative binding affinity (K_{D}) to human pCTS-L (“Hp”) or murine pCTS-L (“Mp”), as well as protective efficacy in CLP sepsis were also noted.

B) Use of 24 peptides corresponding to different region of human pCTS-L to characterize the epitope profile of monoclonal antibodies raised against human pCTS-L. Note that three P13-reactive mAbs recognized both recombinant human pCTS-L (Hp) and murine pCTS-L (Mp) on the dot blots.

FIG. 12A-12B. Protective mAb attenuated the pCTS-L-induced production of cytokines and chemokines in human PBMCs.

A) Recombinant pCTS-L expressed in human HEK293 kidney cells similarly activated human PBMCs. Human PBMCs were isolated from blood of normal healthy subjects and stimulated with recombinant pCTS-L expressed either in human HEK293 kidney cell line or E. coli for 16 h, and extracellular levels of various cytokines and chemokines were measured by Cytokine Antibody Arrays.

B) A protective mAb effectively inhibited pCTS-L-induced cytokines and chemokines in human PBMCs. Human PBMCs were stimulated with recombinant human pCTS-L in the absence or presence of irrelevant control antibodies (c-mAb) or a pCTS-L-neutralizing mAb (mAb20) for 16 h, and extracellular concentrations of cytokines and chemokines were determined by Cytokine Antibody Arrays. Note that pCTS-L induced the release of several cytokines and chemokines, which were effectively inhibited by a protective monoclonal antibody (mAb20), but not irrelevant control (c-mAb).

FIG. 13A-13B. A protective anti-pCTS-L mAb markedly inhibited pCTS-L interaction with TLR4 and RAGE receptors.

A) Effect of a protective mAb on pCTS-L-receptor interactions. Recombinant pCTS-L was immobilized on the NTA sensor chip, and an irrelevant control mAb (c-mAb) or an anti-pCTS-L protective mAb (mAb20) was separately pre-incubated with pCTS-L-conjugated sensor chip before subsequent application of recombinant TLR4 (Left Panel) or RAGE (Right Panel) at increasing concentrations to estimate the K_{D}.

B) Protein-protein docking of pCTS-L/receptor complexes. We used the ClusPro Web Server to predict possible structures of pCTS-L/receptor complexes that exhibited the least Gibbs free energy. The epitope sequence for protective mAbs was marked on pCTS-L (as P13 in purple) to illustrate its possible position within the pCTS-L/receptor complexes. Note the epitope sequence (P13) for protective mAbs was sequestered into the hydrophobic crevices of TLR4 (Left Panel), but positioned sideways in close proximity to the V-domain of RAGE (Right Panel).

FIG. 14A-14B. Anti-pCTS-L mAb conferred a significant protection against CAIA-induced arthritis in mice.

A) Experimental Scheme. Collagen antibody-induced arthritis (CAIA) was induced in Balb/C mice (14-16 weeks, 21-28 g) by administering a cocktail of anti-collagen type II mAbs (α-CII, Cat. #53010, Chondrex; 1.4-1.6 mg/mouse) on day 0, followed by an endotoxin challenge (25-30 μg/mouse) 3 days later. Starting on day 6 post initial α-CII challenge, mice were intraperitoneally administered with either 0.9% saline (as control vehicle) or a pCTS-L-neutralizing mAb (2.0 mg/kg) daily for 5 consecutive days. Animals were carefully examined daily to score the severity of rheumatoid arthritis (up to 4 points per joint, maximum=16 points per mouse) according the following criteria: 0, no signs of redness (erythema) and swelling; 1, erythema and mild swelling confined to one type of joint (the tarsals or ankle joint); 2, erythema and mild swelling extending from the ankle to the tarsals thereby affecting two types of joint; 3, erythema and moderate swelling extending from the ankle to metatarsal joints thereby affecting all three type of joint (entire paw); 4, erythema and severe swelling encompass the ankle, foot and digits, or ankylosis of the limb thereby leading to the disappearance of anatomical definition.

B) Therapeutic efficacy of an anti-pCTS-L mAb 2H8A2 in CAIA-induced arthritis. *, P<0.05 versus control vehicle group, one-way ANOVA.

FIG. 15. Proposed model for pCTS-L-neutralizing mAbs protection against lethal sepsis.

Pathogen-associated molecular pattern molecules (PAMPs such as bacterial lipopolysaccharide, LPS) relies on cell surface pattern recognition receptors (PRR, TLR4) to activate innate immune cells to immediately release “early” proinflammatory mediators (such as TNF, IL-10, and IFN-γ), which then stimulate hepatocytes and innate immune cells to synthesize and secrete a proinflammatory mediator, serum amyloid A (SAA). SAA then activate innate immune cells to upregulate and secrete procathepsin-L (pCTS-L), which binds to cell surface PRRs such as TLR4 and RAGE to induce: i) the expression of cytokines/chemokines to trigger dysregulated inflammation; and ii) the expression of pro-Casp-11 to activate inflammasome and pyroptosis. The pCTS-L-mediated dysregulation inflammation and pyroptosis-associated immunosuppression may adversely contribute to the pathogenesis of lethal sepsis. A panel of P13-reactive mAbs, such as mAb20, could bind to pCTS-L to interrupt its interaction with TLR4 and RAGE, thereby impairing pCTS-L-mediated dysregulated inflammation to confer protection against lethal sepsis.

FIG. 16. Amino acid sequence of human pre-cathepsin L (SEQ ID: NO 2).

FIG. 17. Epitope sequence of human and murine pCTS-L protein for all claimed monoclonal antibodies (SEQ ID NO: 5 and SEQ ID NO: 6).

FIG. 18. Clone 2H8A2 (mAb2) heavy chain DNA Sequence (SEQ ID NO: 7).

FIG. 19. Clone 2H8A2 (mAb2) heavy chain amino acid Sequence (SEQ ID NO: 8).

FIG. 20. Clone 2H8A2 (mAb2) light chain DNA sequence (SEQ ID NO: 9).

FIG. 21. Clone 2H8A2 (mAb2) light chain amino acid sequence (SEQ ID NO: 10).

FIG. 22. Clone 20D5H6 (mAb20) heavy chain DNA Sequence (SEQ ID NO: 11).

FIG. 23. Clone 20D5H6 (mAb20) heavy chain amino acid Sequence (SEQ ID NO: 12).

FIG. 24. Clone 20D5H6 (mAb20) light chain DNA sequence (SEQ ID NO: 13).

FIG. 25. Clone 20D5H6 (mAb20) light chain amino acid sequence (SEQ ID NO: 14).

FIG. 26. Clone 26C7C9 (mAb26) heavy chain DNA Sequence (SEQ ID NO: 15).

FIG. 27. Clone 26C7C9 (mAb26) heavy chain amino acid Sequence (SEQ ID NO: 16).

FIG. 28. Clone 26C7C9 (mAb26) light chain DNA sequence (SEQ ID NO: 17).

FIG. 29. Clone 26C7C9 (mAb26) light chain amino acid sequence (SEQ ID NO: 18).

FIG. 30. Comparison of CDR regions of three P13-reacting mAbs (from SEQ ID NOs: 8, 10, 12, 14, 16 and 18) and their binding affinities (K_{D}) for murine or human pCTS-L protein.

FIG. 31. Comparison of amino acid sequence of three P13-reacting and protective mAbs (SEQ ID NOs: 8, 10, 12, 14, 16 and 18).

### DETAILED DESCRIPTION OF THE INVENTION

The invention provides monoclonal antibodies that specifically binding to amino acid sequence GGLDSEESYPYEATEESCKYN (SEQ ID NO: 6) present in residues 194-214 of human precathepsin L (SEQ ID NO: 2), or an antigen-binding fragment thereof, comprising a heavy chain variable region (VH) and a light chain variable region (VL), wherein
(A)
   (i) the heavy chain variable region (VH) comprises: (part of SEQ ID NO: 8)
   (a) an amino acid sequence of heavy chain CDR1 comprising the amino acid sequence DTYMH (SEQ ID NO: 31);
   (b) an amino acid sequence of heavy chain CDR2 comprising the amino acid sequence RIDPAIVNTRYDPKFQD (SEQ ID NO: 32); and
   (c) an amino acid sequence of heavy chain CDR3 comprising the amino acid sequence LGNYYGSRYVMDY (SEQ ID NO: 33); and
   (ii) the light chain variable region (VL) comprises: (part of SEQ ID NO: 10)
   (a) an amino acid sequence of light chain CDR1 comprising the amino acid sequence RSSQSLVHSNGNTYLH (SEQ ID NO: 34);
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPYT (SEQ ID NO: 36);
   or
(B)
   (i) the heavy chain variable region (VH) comprises: (part of SEQ ID NO: 12)
   (a) an amino acid sequence of heavy chain CDR1 comprising the amino acid sequence NTYMH (SEQ ID NO: 37);
   (b) an amino acid sequence of heavy chain CDR2 comprising the amino acid sequence RIDPTNGNTKYAPKFQG (SEQ ID NO: 38); and
   (c) an amino acid sequence of heavy chain CDR3 comprising the amino acid sequence GDYGDGAY (SEQ ID NO: 39); and
   (ii) the light chain variable region (VL) comprises: (part of SEQ ID NO: 14)
   (a) an amino acid sequence of light chain CDR1 comprising the amino acid sequence RSSQSLVHSNGNTYLH (SEQ ID NO: 34);
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPYT (SEQ ID NO: 36);
   or
(C)
   (i) the heavy chain variable region (VH) comprises: (part of SEQ ID NO: 16)
   (a) an amino acid sequence of heavy chain CDR1 comprising the amino acid sequence NTYMY (SEQ ID NO: 40);
   (b) an amino acid sequence of heavy chain CDR2 comprising the amino acid sequence RIDPANGNIKYDPKFQG (SEQ ID NO: 41); and
   (c) an amino acid sequence of heavy chain CDR3 comprising the amino acid sequence RKVMTADYFDY (SEQ ID NO: 42); and
   (ii) the light chain variable region (VL) comprises: (part of SEQ ID NO: 18)
   (a) an amino acid sequence of light chain CDR1 comprising the amino acid sequence RSSQSLVHSNGNTYLH (SEQ ID NO: 34):
   (b) an amino acid sequence of light chain CDR2 comprising the amino acid sequence KVSNRFS (SEQ ID NO: 35); and
   (c) an amino acid sequence of light chain CDR3 comprising the amino acid sequence SQSTHVPWT (SEQ ID NO: 43).

Antibody (A) above can be derived from mouse monoclonal antibody clone #2H8A2 (mAb2). Antibody (B) can be derived from mouse monoclonal antibody clone #20D5H6 (mAb20). Antibody (C) can be derived from mouse monoclonal antibody clone #26C7C9 (mAb26).

Preferably, the antibody is a humanized antibody. Preferably, the class (subclass) of the humanized antibody is IgG1(λ) or IgG2(λ).

Preferably, (i) the heavy chain variable region (VH) comprises the amino acid sequences of SEQ ID NOs: 8, 12 and 16 as amino acid sequences of FR1, FR2, FR3 and FR4, respectively; and (ii) the light chain variable region (VL) comprises the amino acid sequences of SEQ ID NOs: 10, 14, and 18 as amino acid sequences of FR1, FR2, FR3 and FR4, respectively.

In one embodiment, (i) the heavy chain variable region (VH) comprises an amino acid sequence derived from the mouse mAb2, mAb20, and mAb26 H chain, respectively, and (ii) the light chain variable region (VL) comprises an amino acid sequence derived from the mouse antibody mAb2, mAb20, and mAb26 L chain, respectively.

In one embodiment, (i) the heavy chain variable region (VH) comprises an amino acid sequence having a 90% or more identity with the amino acid sequence of SEQ ID NO: 8, 12, and 16, respectively and (ii) the light chain variable region (VL) comprises an amino acid sequence having a 90% or more identity with the amino acid sequence of SEQ ID NO: 10, 14, and 18, respectively.

Also provided is a monoclonal antibody that specifically binds to amino acid sequence GGLDSEESYPYEATEESCKYN (SEQ ID NO: 6), wherein the monoclonal antibody has at least 90% sequence identify to the monoclonal antibody described herein above.

In one embodiment, the antibody or antigen-binding fragment thereof, has a binding activity to human pCTS-L protein (analyzed by OpenSPR) that is 10-fold lower than that of murine pCTS-L.

Also provided are pharmaceutical compositions comprising the humanized antibody or antigen-binding fragment thereof described herein and a pharmaceutically acceptable carrier. As used herein, a “pharmaceutically acceptable carrier” is (i) compatible with the other ingredients of the composition without rendering the composition unsuitable for its intended purpose, and (ii) suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include any of the standard pharmaceutical carriers such as phosphate buffered saline solutions, water, and emulsions such as oil/water emulsions and microemulsions. The pharmaceutical compositions can be used in treatment or prevention of various diseases where pCTS-L is pathologically overexpressed, such as, for example, sepsis, rheumatoid arthritis, and other inflammatory diseases such as COVID-19.

Also provided is an isolated nucleic acid encoding the amino acid sequence of the antibody or antigen-binding fragment, as described in SEQ ID NOs 7, 9, 11, 13, 15, or 17, or an isolated nucleic acid hybridizable with any of these nucleic acids under high stringent conditions. Also provided are recombinant expression vectors comprising the isolated nucleic acid and host cells transformed with the recombinant expression vector.

Also provided are methods of treating, for example, sepsis, rheumatoid arthritis or an inflammatory disease in a patient in need thereof comprising administrating to the patient a therapeutically effective amount of the antibody or antigen-binding fragment. The inflammatory disease can be, for example, one or more of pancreatitis, atherosclerosis, chronic kidney disease, end-of stage renal disease, vascular injury, antigen-induced arthritis, dextran sulfate sodium (DSSD)-induced colitis and Covid-19. The inflammatory disease can be, for example, one or more of Inflammatory conditions treated or attenuated by reducing circulating cytokine levels in a subject in need thereof include appendicitis, peptic, gastric or duodenal ulcers, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, Alzheimer's disease, meningitis, encephalitis, multiple sclerosis, cerebral infarction, cerebral embolism. Guillarne-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, ankylosing spondylitis, Type I diabetes, ankylosing spondylitis, Berger's disease, reactive arthritis (Reiter's syndrome) or Hodgkin's disease. In more preferred embodiments, the condition is appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection and graft-versus-host disease.

### Experimental Details

### Overview

For the treatment of sepsis, antibody-based strategies have only been attempted to antagonize early pro-inflammatory cytokines, but not yet been tried for other inducible late-acting mediators. Here we develop an antibody strategy to target a novel late cytokine, procathepsin-L (pCTS-L), in pre-clinical settings. The expression and secretion of pCTS-L was induced by an “intermediate” inflammatory mediator, human serum amyloid A (SAA) in innate immune cells, contributing to its sustained and systemic accumulation in experimental and clinical sepsis. Recombinant pCTS-L effectively induced IL-6, IL-8, GRO-α/KC, GRO-β/MIP-2 and MCP-1 in innate immune cells, and positively correlated with the blood concentrations of these cytokines/chemokines in clinical sepsis. Mechanistically, pCTS-L interacted with the toll-like receptor 4 (TLR4) as well as the receptor for advanced glycation end products (RAGE) to induce cytokines/chemokines, Casp-11, and tissue injury. Pharmacological administration of pCTS-L protein, pCTS-neutralizing polyclonal antibodies or genetic disruption of Ctsl expression distinctly affected the outcomes of lethal endotoxemia and sepsis, revealing a previously under-appreciated pathogenic role for extracellular pCTS-L in sepsis. Furthermore, we developed a panel of murine and human pCTS-L-neutralizing monoclonal antibodies that effectively attenuated pCTS-L-mediated inflammation, and rescued animals from lethal sepsis. Furthermore, these pCTS-L-neutralizing mAbs similarly attenuated the progression of rheumatoid arthritis in a murine model of anti-collagen antibody-induced arthritis. Here we report the development and evaluation of a panel of pCTS-L-neutralizing mAbs using animal models of lethal sepsis and arthritis.

Identification of pCTS-L as an SAA-Inducible and Secretory Protein.

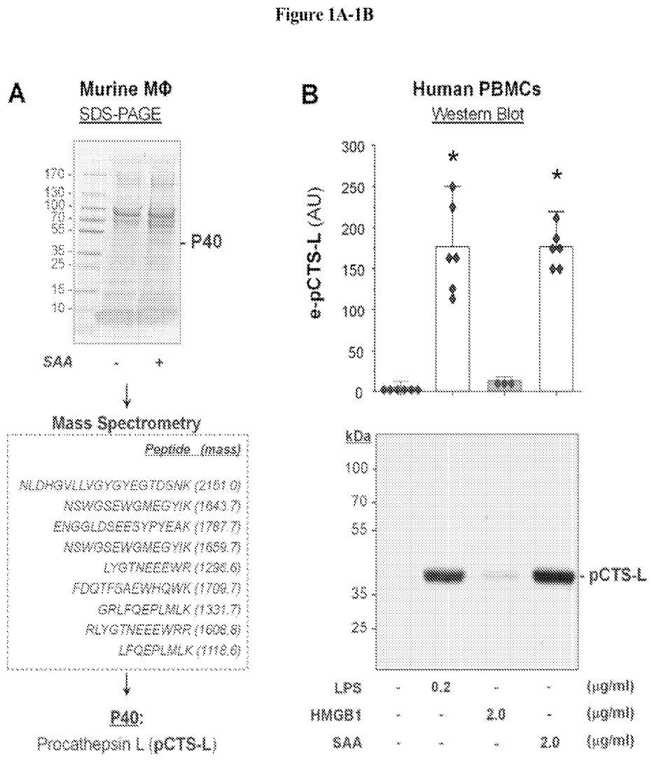

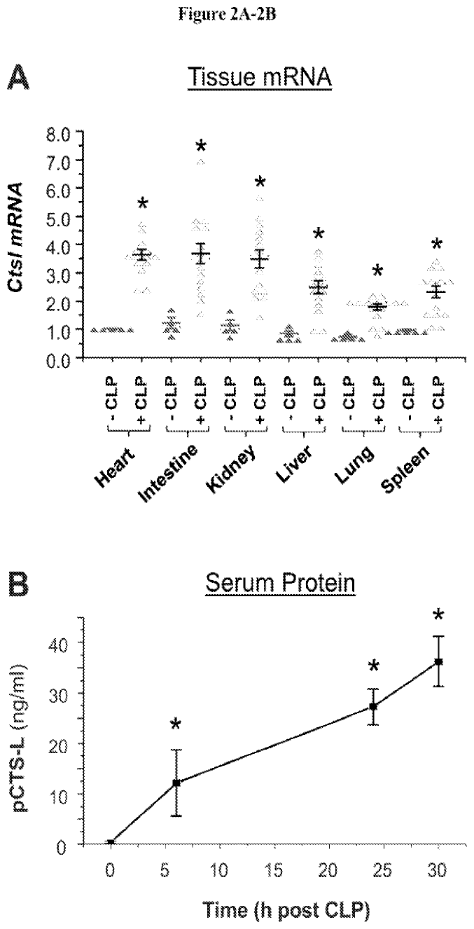

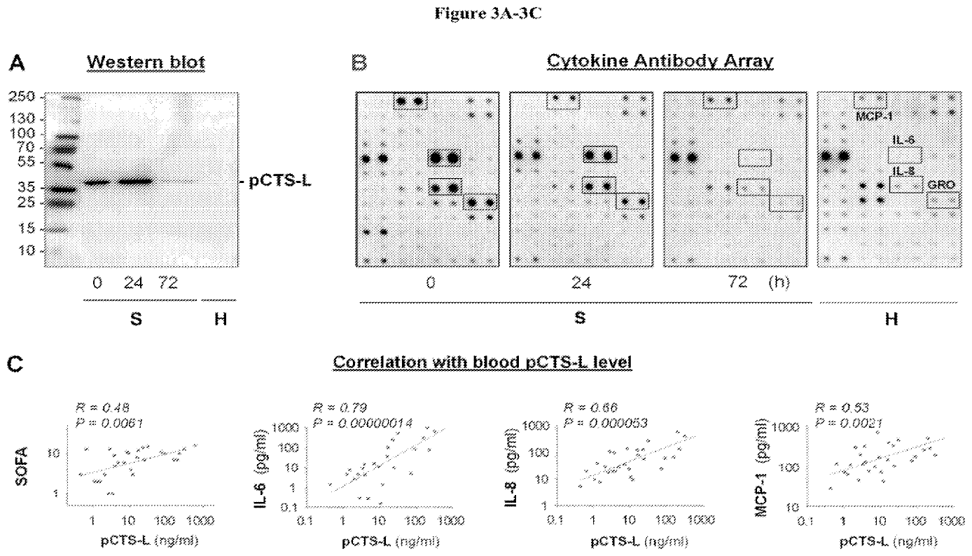

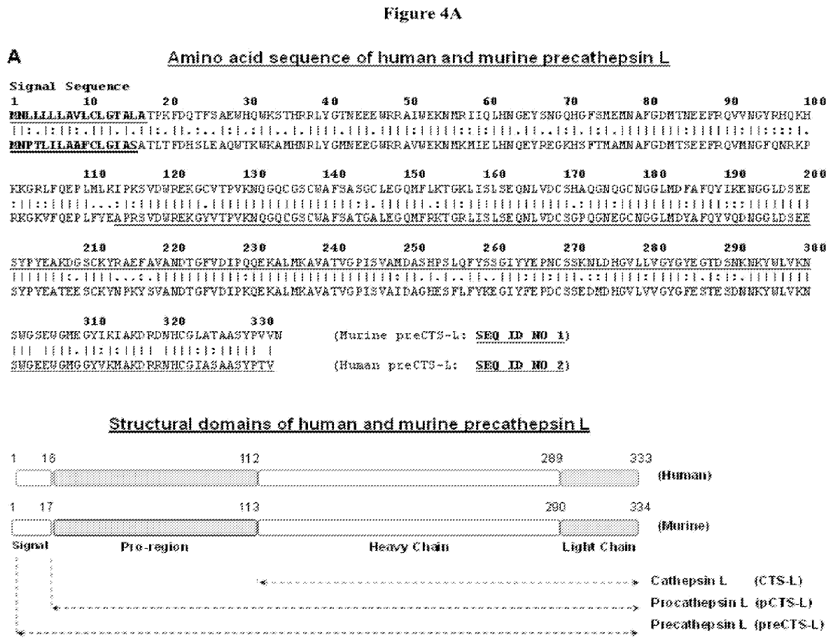

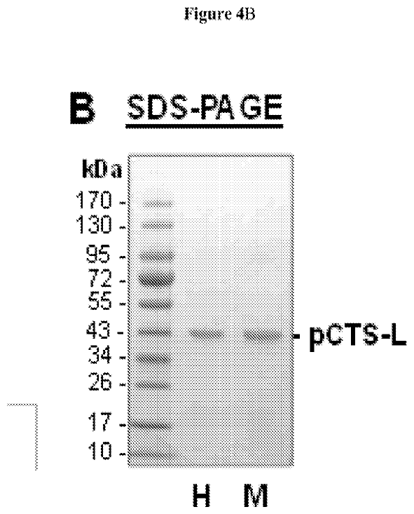

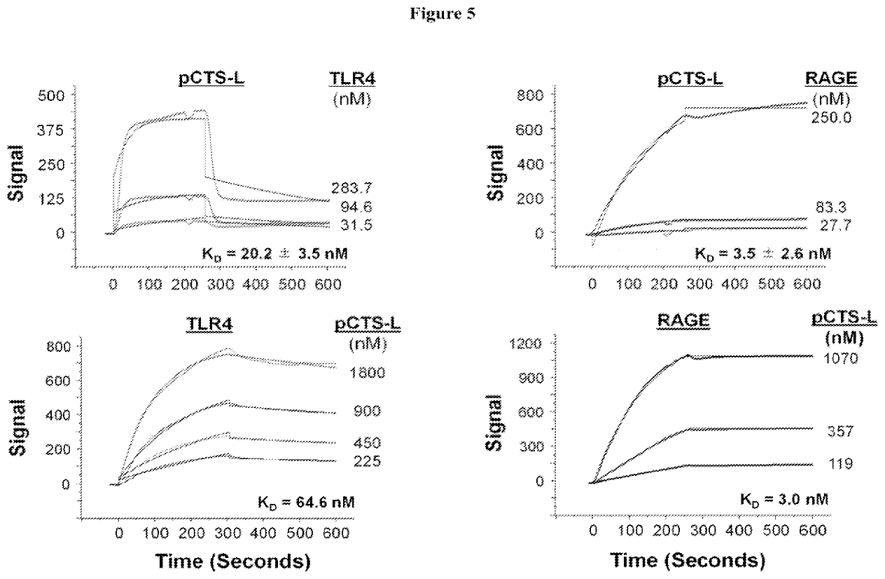

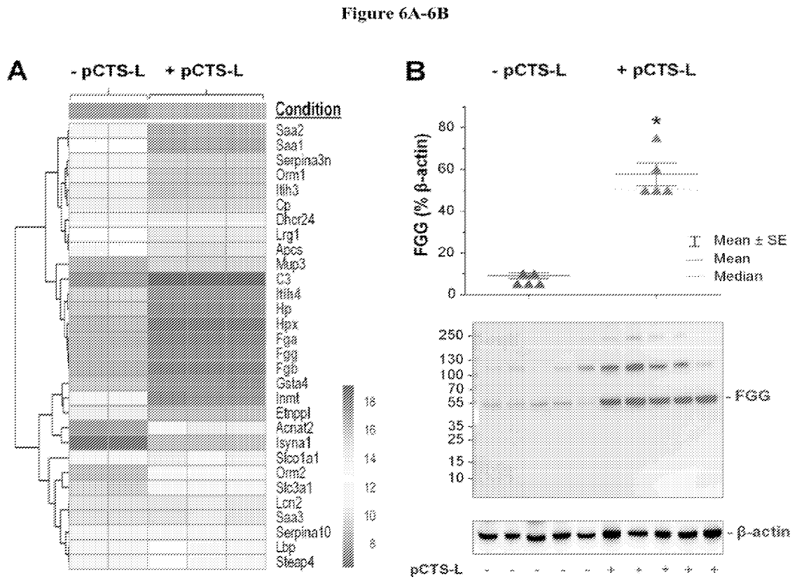

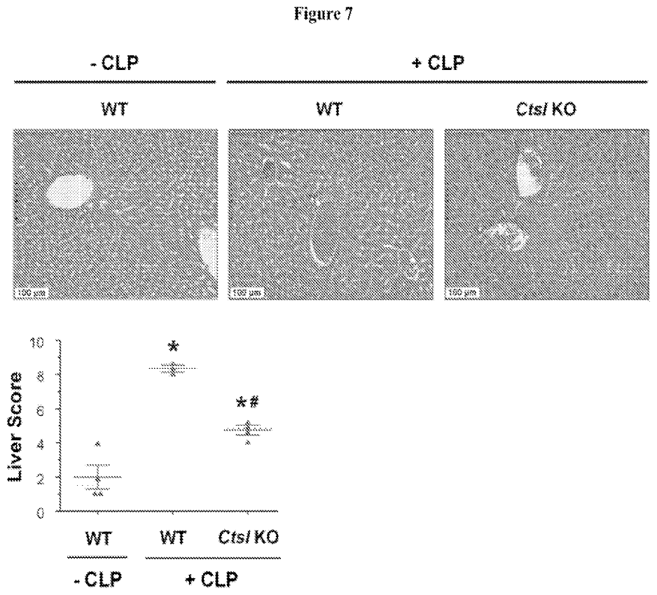

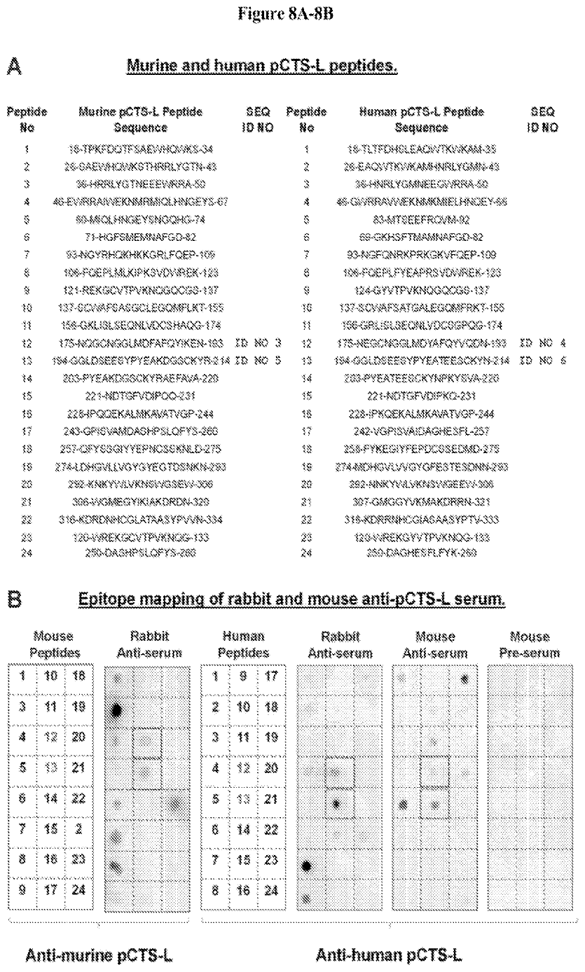

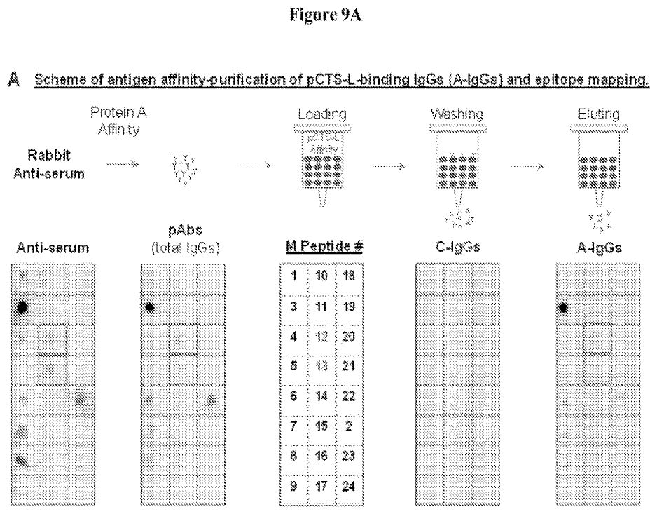

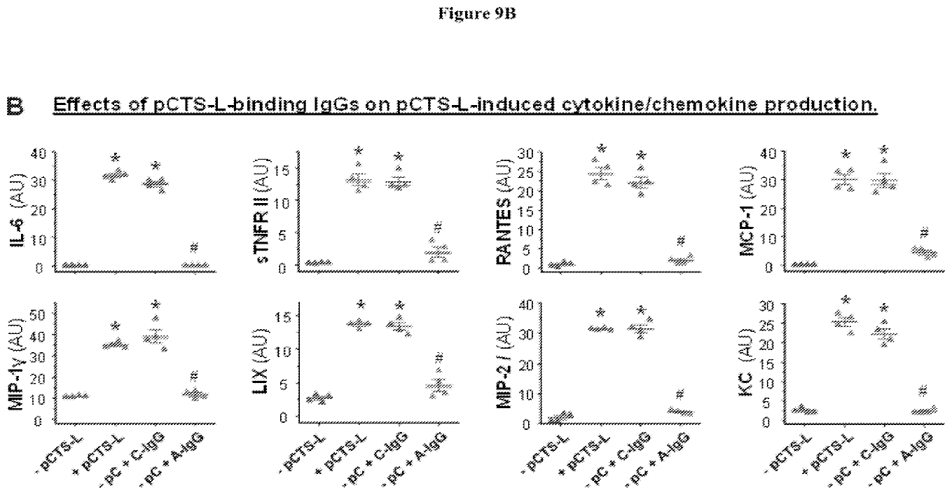

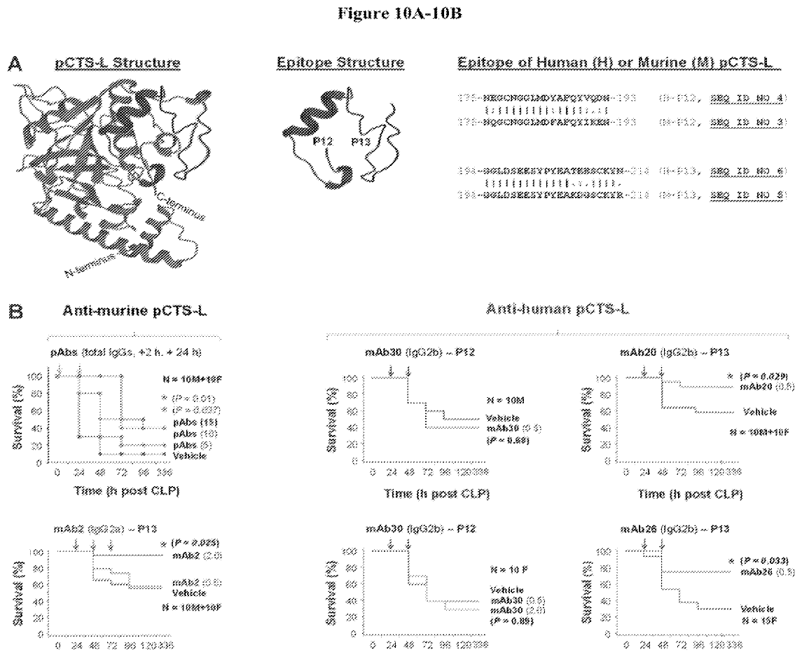

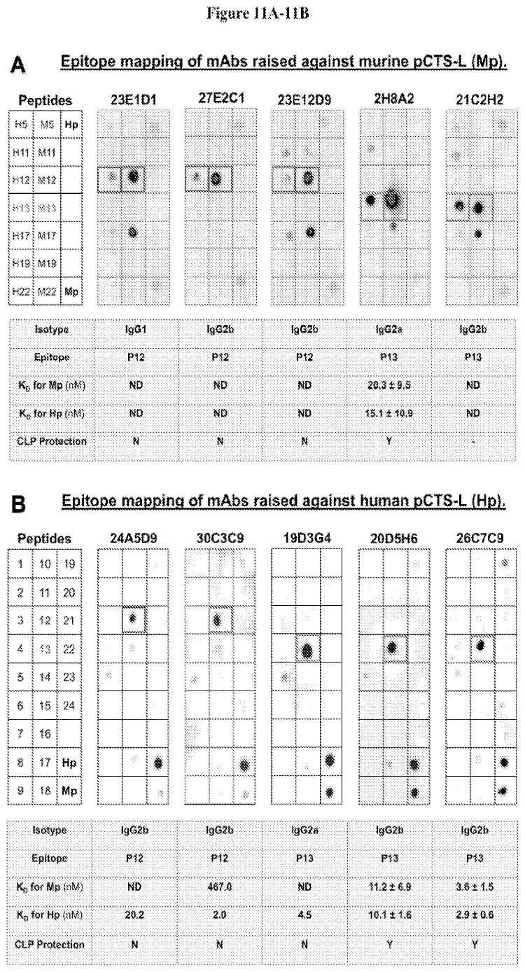

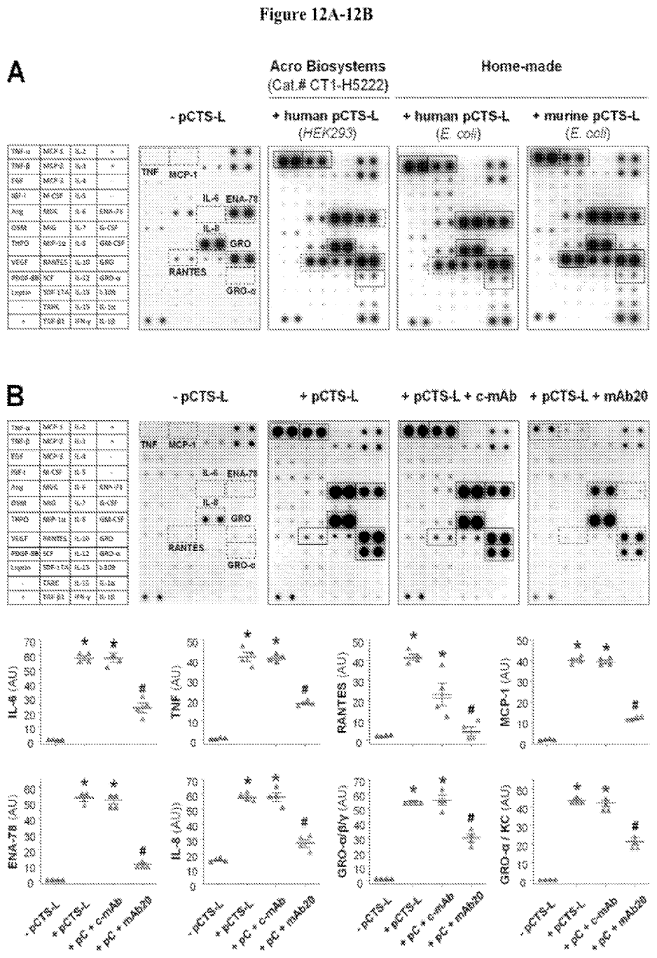

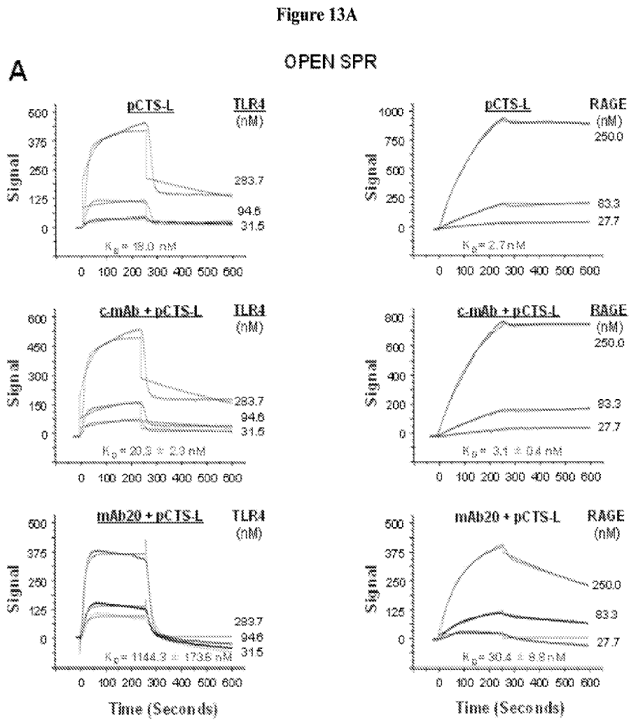

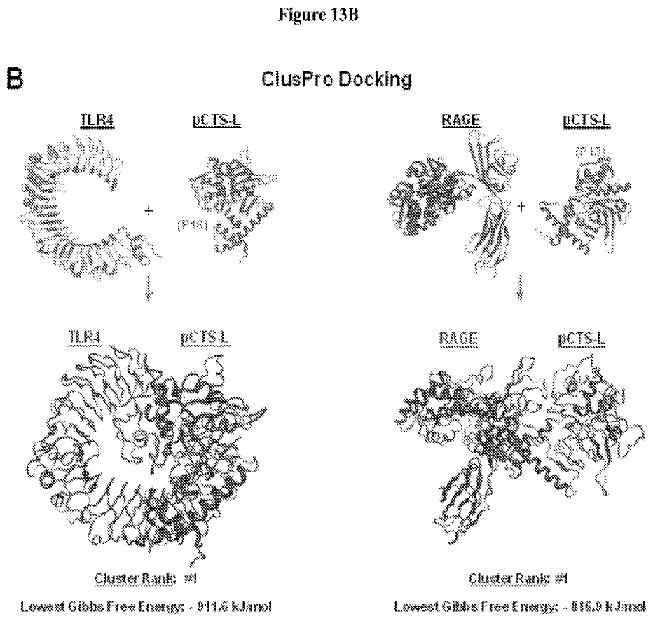

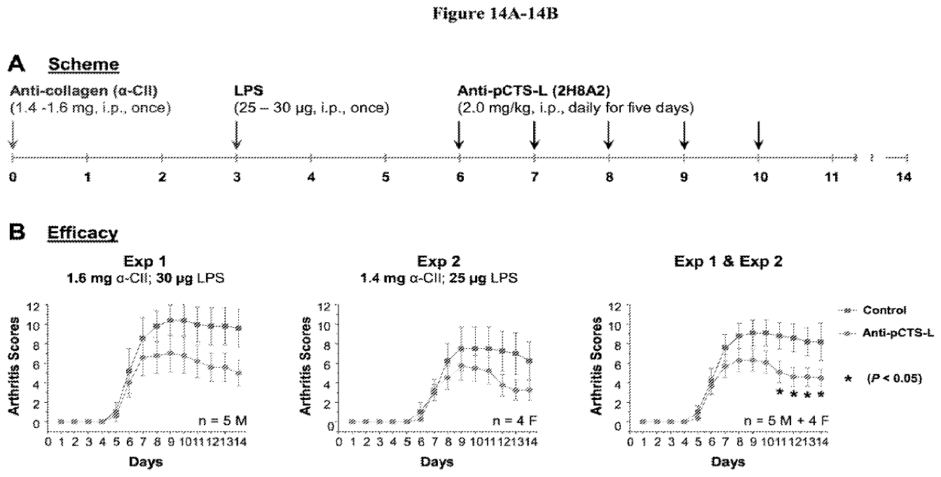

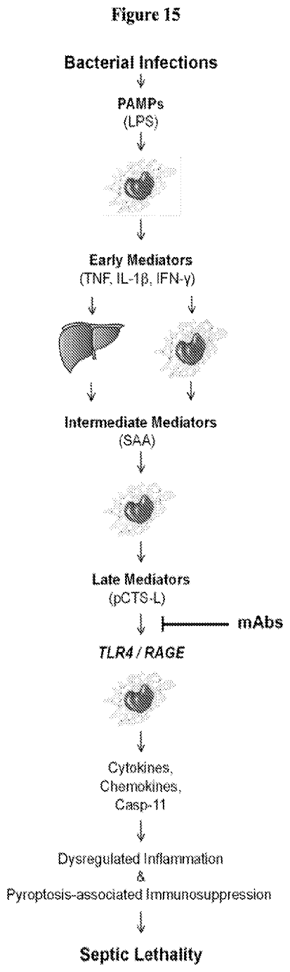

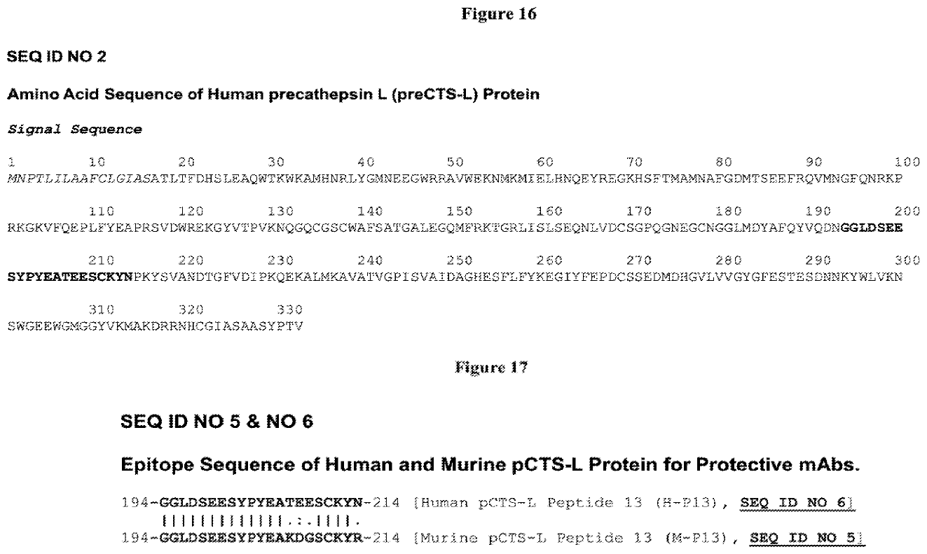

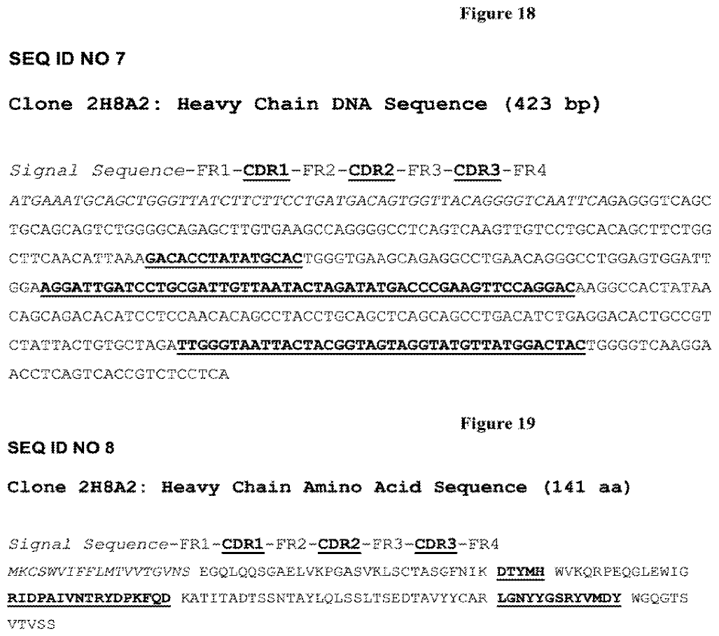

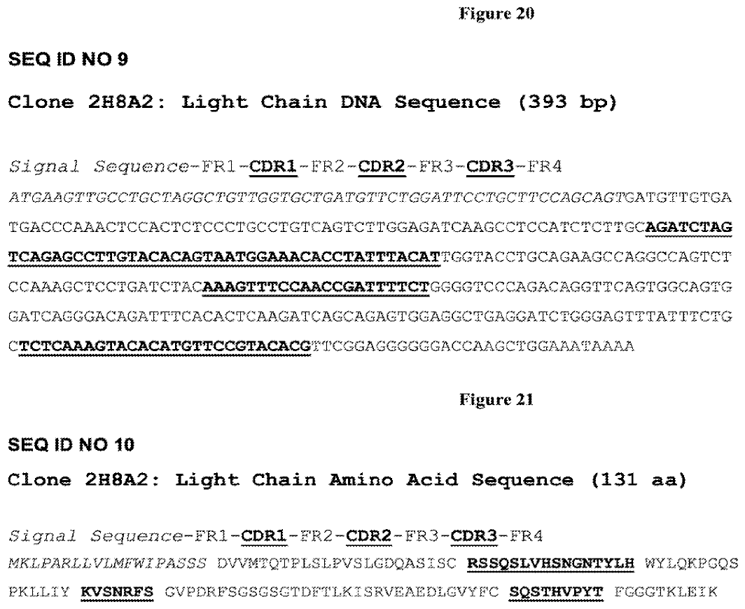

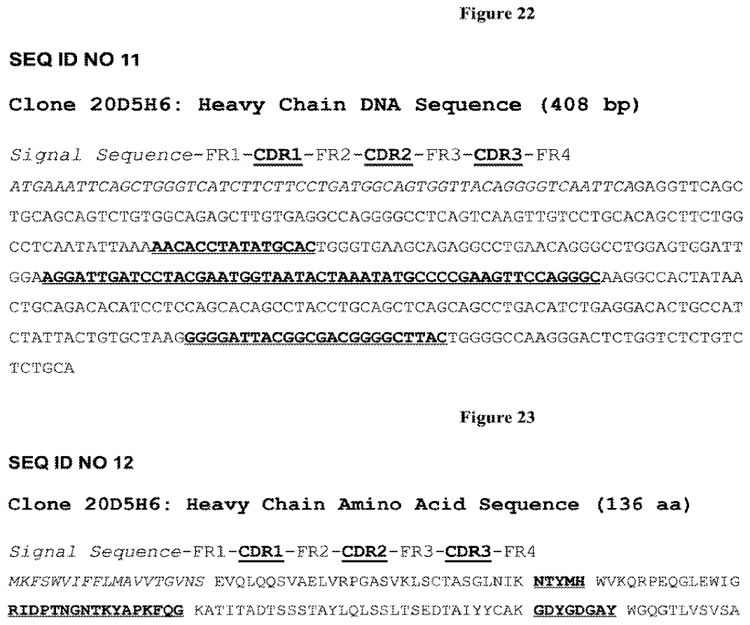

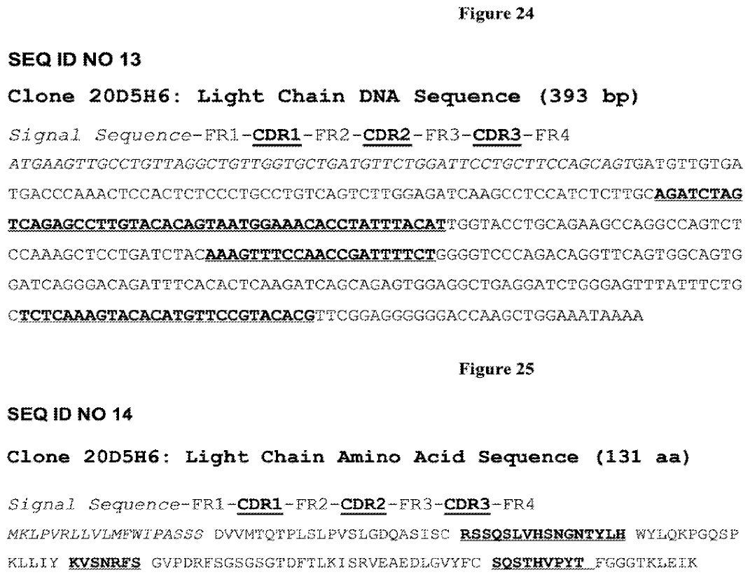

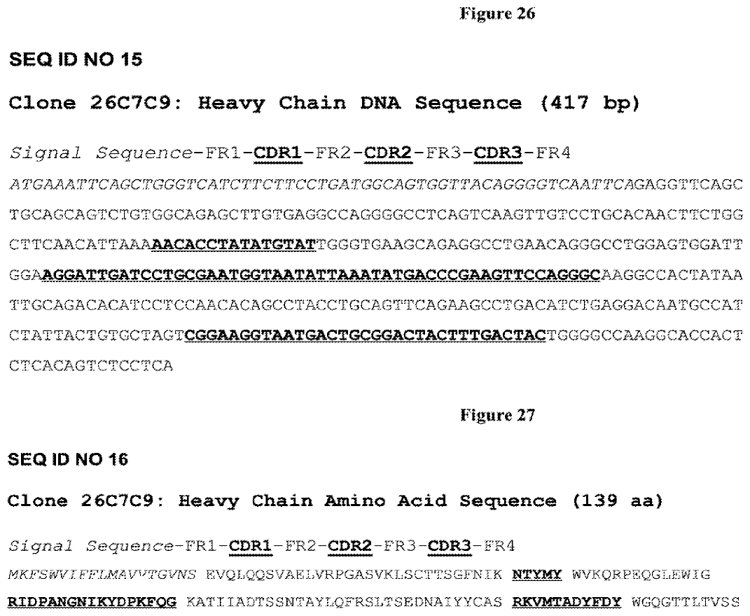

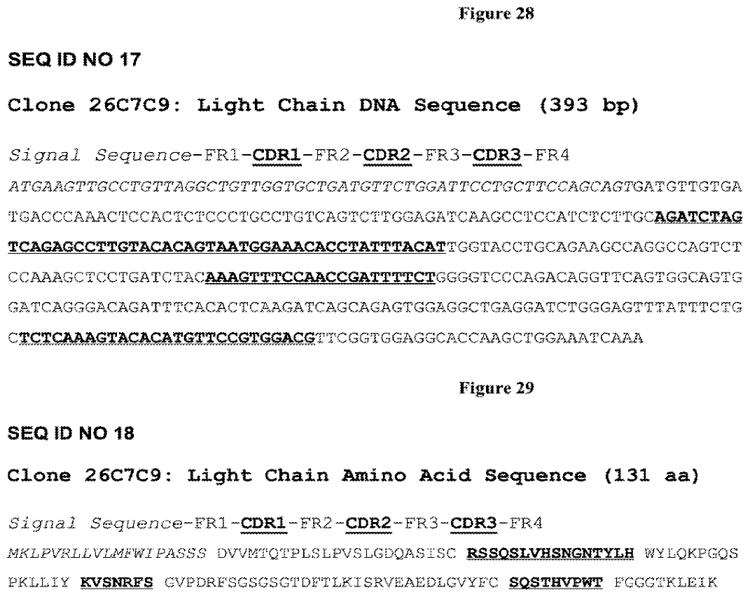

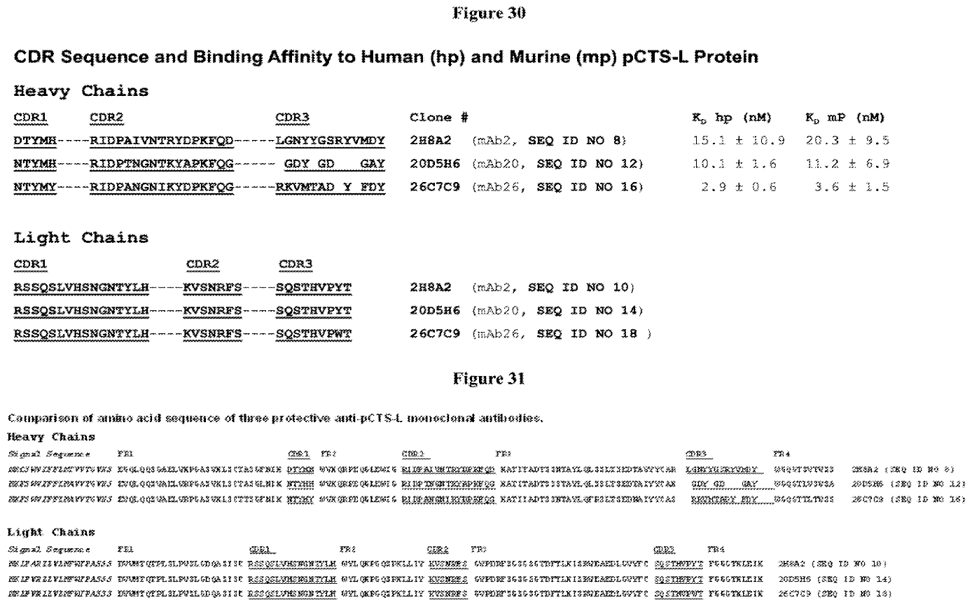

To search for late-acting mediators of sepsis, we characterized SAA-inducible proteins in murine macrophage-conditioned culture medium. Prolonged stimulation with SAA resulted in a marked elevation of the extracellular content of a 40-kDa protein (“P40”, FIG. 1A), which was identified as murine pCTS-L by in-gel trypsin digestion and mass spectrometry (FIG. 1A). To verify its identity, the culture medium conditioned by SAA- or LPS-stimulated macrophages was immunoblotted with a pCTS-L-specific mAb, which confirmed a marked pCTS-L release in response to SAA or LPS stimulation. In addition, we stimulated human primary peripheral blood mononuclear cells (PBMCs) with LPS, SAA or HMGB1 for 16 h, and measured pCTS-L content in PBMC-conditioned medium by Western blotting analysis (FIG. 1B). The amounts of extra-cellular pCTS-L were similarly elevated by LPS and SAA, but not by HMGB1 (FIG. 1B), suggesting that both microbial PAMPs and host proinflammatory mediators induced pCTS-L expression and secretion in innate immune cells.

Up-Regulation and Systemic Accumulation of pCTS-L in Experimental Sepsis.

To confirm its up-regulation in vivo, we employed real-time RT-PCR to survey the mRNA expression level of Ctsl in septic animals (FIG. 2A). The expression of Ctsl mRNA was uniformly elevated in the heart, intestine, kidney, liver, lung, and spleen (FIG. 2A), suggesting that Ctsl was predominantly upregulated in experimental sepsis. These findings were consistent with a previous report that Ctsl was upregulated by 3-folds in the skeletal muscle as early as 2 days post E. coli infection (42). To assess its kinetics of systemic accumulation, we measured circulating pCTS-L protein concentrations at several time points post CLP. Circulating pCTS-L was not detected in normal healthy animals, but time-dependently elevated in septic animals, approaching plateau at 24-32 h post CLP (FIG. 2B), a time period when some septic animals started to succumb to death. It suggested a relatively late and systemic pCTS-L accumulation in a pre-clinical model of sepsis.

Systemic Accumulation of pCTS-L and Other Surrogate Biomarkers in Clinical Sepsis.

To evaluate its clinical relevance, we employed various immunoassays to characterize the dynamic changes of circulating pCTS-L in parallel with several surrogate markers of clinical sepsis. Although pCTS-L was not detected in the plasma of normal healthy controls (“H”, FIG. 3A), it was significantly elevated in age- and sex-matched septic patients at 0-72 h after their clinical diagnosis (FIG. 3A). In patients with clinical sepsis, the plasma pCTS-L contents also positively correlated with corresponding SOFA scores (FIG. 3C) and the blood concentrations of several cytokines (IL-6) and chemokines (IL-8 and MCP-1. FIG. 3C) previously characterized as surrogate markers of experimental (43, 44) and clinical sepsis (45). These findings have confirmed a sustainably late and systemic accumulation of pCTS-L in pre-clinical and clinical settings.

Requirement of TLR4 and RAGE Receptors for pCTS-L-Induced Dysregulated Inflammation.

In light of the TLR4 dependence of a distantly related liver fluke pCTS-L in activating dendritic cells (27), we generated recombinant human and murine pCTS-L (FIG. 4A, 4B; SEQ ID NO: 1 and SEQ ID NO: 2) to verify their possible interaction with TLR4 and other receptors using the Open Surface Plasmon Resonance (OpenSPR). Recombinant pCTS-L was purified to high homogeneity by affinity chromatography (FIG. 4B), followed by extensive Triton X-114 extractions to remove contaminating endotoxins. When highly purified human pCTS-L was immobilized onto a sensor chip and human TLR4 or RAGE was respectively applied as analytes at various concentrations, pCTS-L exhibited high affinities to both TLR4 (with an estimated equilibrium dissociation constant, K_{D}=20.2±3.5 nM, FIG. 5) and RAGE (K_{D}=3.5±2.6 nM, FIG. 5). Conversely, when TLR4 or RAGE receptor was respectively conjugated to a sensor chip and pCTS-L ligand was applied as analyte, pCTS-L exhibited a similarly strong interaction with TLR4 (K_{D}=64.4 nM, FIG. 5) and RAGE (K_{D}=3.0 nM, FIG. 5), suggesting a possibility that pCTS-L might elicit dysregulated inflammation through these PRR receptors.

To test this possibility, we first determined whether genetic disruption of TLR4 and RAGE expression impaired pCTS-L-induced dysregulated inflammation. The disruption of TLR4 alone markedly reduced the pCTS-L-induced secretion of most cytokines (e.g., IL-6 and IL-12) and chemokines (e.g., RANTES, MCP-1, MIP-1γ and LIX) except for MIP-2/GRO-0 and KC/GRO-α, two neutrophilic surrogate markers of experimental sepsis (43). However, the double knockout of both TLR4 and RAGE resulted in a complete abolishment of all pCTS-L-induced cytokines and chemokines that included KC/GRO-α and MIP-2/GRO-β, confirming an important role for both TLR4 and RAGE in pCTS-L-mediated dysregulated inflammation in vivo.

Excessive inflammation is often accompanied by a parallel up-regulation and activation of Casp-11/4/5, which triggers dysregulated inflammasome activation, pyroptosis, and passive DAMP release (12). Interestingly, pCTS-L dose-dependently induced pro-Casp-11 expression and Casp-11 maturation in peritoneal macrophages derived from wildtype but not from TLR4/RAGE-deficient mice. Similarly, pCTS-L dose-dependently induced the release of SQSTM1 from wildtype but not TLR4/RAGE-deficient macrophages. Collectively, these findings support an important role for TLR4 and RAGE in pCTS-L-mediated dysregulated inflammation as well as Casp-11-associated dysregulated pyroptosis and immunosuppression.

Requirement of TLR4 and RAGE Receptor for pCTS-L-Induced Tissue Injury.

To elucidate the role of pCTS-L in sepsis, we intraperitoneally injected mice with recombinant pCTS-L at suprapathological doses, and found that pCTS-L caused marked injuries to the liver (i.e., the increase in hepatocellular necrosis, cytoplasmic vacuolization and sinusoidal congestion) and intestine (i.e., the loss of villi). Notably, the pCTS-L-induced tissue injuries were more rigorous in wildtype C57BL/6 mice as compared with mutant C57BL/6 mice deficient in both TLR4 and RAGE, supporting an important role for these PRR receptors in pCTS-L-mediated tissue injuries. To further elucidate the mechanisms underlying pCTS-L-mediated pathogenesis, normal healthy Balb/C mice were intraperitoneally administered with recombinant pCTS-L, and various tissues were harvested to characterize the expression profile of a full catalog of transcripts by RNA Sequencing (RNA-Seq). Administration of pCTS-L markedly upregulated several genes involved in the acute-phase response (e.g., SAA1/2/3, Hp and Hpx) and coagulation (e.g., Fga, Fgb, and Fgg) in the liver (FIG. 6A). For instance, administration of pCTS-L induced a marked increase in hepatic mRNA expression (FIG. 6A) and protein content (FIG. 6B) of fibrinogen-γ (FGG), a substrate for thrombin-catalyzed production of fibrin, which can aggregate to form 3-dimensional structural network capable of binding platelets and trapping blood cells to form thrombus (46). However, the possible roles of other altered genes in the pathogenesis of pCTS-L-mediated tissue injury remains an exciting subject of future studies.

### Genetic Deletion of Ctsl Conferred Protection Against Sepsis-Induced Tissue Injury.

To confirm the pathogenic role of pCTS-L in sepsis, we first attempted the genetic gene-knockout approach by obtaining a few breeding pairs of heterozygous Ctsl^{+/-} KO mice. Unfortunately, female Ctsl^{-/-} KO were found infertile, because CTS-L was still needed for the normal degradation of the follicle wall to release mature oocytes during the process of ovulation (47). Similarly, male Ctsl^{-/-} KO mice might also suffer from poor reproductivity, because mutant mice expressing inactive CTS-L was also found abnormal in spermatogenesis (48). Therefore, we bred heterozygous Ctsl^{+/-} mice to produce a limited number of genetic background-, age-, and sex-matched wild-type littermates (Ctsl^{+/+}) and pCTS-L-deficient (Ctsl^{-/-}) mice. The genotypes of wild-type littermates and Ctsl knock-out (KO) mice were confirmed by genotyping and immunoblotting of tail and serum samples, respectively. Consistent with previous findings that genetic deletion of Ctsl led to an attenuation of pancreatitis (49), atherosclerosis (50), intimal hyperplasia (51) and antigen-induced arthritis (41), we found that genetic disruption of pCTS-L similarly lessened CLP-induced injury to the liver (FIG. 7), confirming a pathogenic role of pCTS-L in lethal sepsis.

pCTS-L-Neutralizing Polyclonal Antibodies (pAbs) Conferred Protection Against Lethal Sepsis.

To confirm its extracellular role in sepsis, we generated pCTS-L-specific pAbs in rabbits (FIG. 8) and tested their pharmacological effects on the lethal outcomes of sepsis. The epitope profiles of anti-pCTS-L rabbit serum were determined by dot blotting with 24 synthetic peptides corresponding to different region of murine or human pCTS-L proteins (FIG. 8A, 8B; FIG. 9A), which revealed the presence of pAbs recognizing two homologous peptides corresponding to residue 175-193 (“P12”; SEQ ID NO: 3 and SEQ ID NO: 4) and residue 194-214 (“P13”; SEQ ID NO: 5 and SEQ ID NO: 6) of murine and human pCTS-L (FIG. 8B, FIG. 9A; FIG. 10A). Anti-murine pCTS-L total IgGs (pAbs) conferred a dose-dependent and significant protection against lethal sepsis in both male and female mice when the first dose was given at 2 h post CLP (FIG. 10B, Top Left Panel). Anti-murine pCTS-L rabbit serum was subjected to Protein-A-affinity chromatography to harvest total IgGs (“PAbs”, FIG. 9A), which was then subjected to pCTS-L-antigen-affinity chromatography to isolate antigen-binding IgGs (A-IgGs) (FIG. 9A). In a sharp contrast to the non-pCTS-L-binding control IgGs (C-IgGs), pCTS-L-antigen-affinity purified IgGs (A-IgGs) completely abrogated the pCTS-L-induced production of cytokines and chemokines (FIG. 9B), which included both the TLR4-dependent IL-6, MIP-1γ, LIX, RANTES and MCP-1, as well as the RAGE-dependent KC/GRO-α and MIP-2/GRO-β. Thus, our findings suggested that anti-pCTS-L pAbs conferred protection against lethal sepsis partly by attenuating pCTS-L-induced dysregulated inflammation orchestrated by both TLR4 and RAGE receptors.

Generation of Monoclonal Antibodies (mAbs) Against Murine and Human pCTS-L.

To develop potential therapies for human sepsis, we generated mAbs against both murine and human pCTS-L and explored their therapeutic potential by administering the first dose of mAb to septic animals in a delayed fashion—starting at 24 h after CLP, a time point when circulating pCTS-L approached plateau concentrations and some septic animals started to succumb to death. Most of the mAbs cross-reacted with two homologous peptides corresponding to residue 175-193 (P12; SEQ ID NO: 3 and SEQ ID NO: 4) or 194-214 (P13; SEQ ID NO: 5 and SEQ ID NO: 6) of murine (FIG. 10A; FIG. 11A) and human pCTS-L (FIG. 10A; FIG. 11B). Notably, mAbs cross-reacting with P12 peptide (e.g., mAb30) did not significantly affect animal survival when given at a wide range of doses (0.5-2.0 mg/kg, FIG. 10B, Middle Panels). In a sharp contrast, three mAbs recognizing P13 peptides of both human and murine pCTS-L effectively rescued animals from lethal sepsis even when the 1^{st} dose was given at 24 h post the onset of sepsis (FIG. 10B, Bottom Left and Right Panels). Consistently, the protective mAb significantly attenuated sepsis-induced systemic accumulation of IL-6, sTNF-RI, and several chemokines (e.g., MIP-1γ, MIP-2/GRO-β and KC/GRO-α), suggesting that these beneficial antibodies confer protection against lethal sepsis possibly by attenuating sepsis-induced dysregulated inflammation.

Anti-pCTS-L mAb Attenuated pCTS-L-Induced Inflammation in Human PBMCs.

To further elucidate the protective mechanisms of pCTS-L-specific mAbs, we examined their effects on pCTS-L-induced production of cytokines/chemokines in primary human PBMCs. Recombinant pCTS-L induced several cytokines (e.g., IL-6 and TNF) and chemokines (e.g., RANTES, MCP-1, ENA-78/LIX, IL-8, GRO-α/KC, and GRO-α/β/γ) in human PBMCs (FIG. 12A). These inflammatory activities were not likely due to contaminating bacterial endotoxins, because extensive extractions of recombinant pCTS-L with Triton-X114 effectively reduced endotoxin content to <0.01 U per microgram protein. Likewise, the endotoxin-free recombinant pCTS-L produced in eukaryotic (HEK293 kidney) cells similarly induced these cytokines/chemokines in human PBMCs (FIG. 12A). Moreover, the pCTS-L-induced production of these cytokines/chemokines were markedly suppressed by the co-addition of a protective anti-pCTS-L mAb (mAb20, FIG. 12B), and not affected by an irrelevant control mAb (c-mAb, FIG. 12B). Collectively, these findings suggest that anti-pCTS-L mAbs confer protection against sepsis possibly by neutralizing the proinflammatory activities of extracellular pCTS-L.

To further understand the neutralizing mechanism of these protective anti-pCTS-L mAbs, we examined their effects on pCTS-L interaction with TLR4 and RAGE receptors. An irrelevant control mAb (c-mAb), did not affect pCTS-L interaction with neither TLR4 nor RAGE when it was pre-incubated with the pCTS-L-conjugated sensor chip even at extremely high concentrations (e.g., 1200 nM, FIG. 13A). In a sharp contrast, a protective anti-pCTS-L mAb (mAb20), markedly reduced pCTS-L's affinities to both receptors, as manifested by an almost 55-fold (from 20.3±2.3 nM to 1144.3±173.6 nM) and 10-fold (from 3.1±0.4 nM to 30.4±9.8 nM) increase in the K_{D} for TLR4 and RAGE, respectively (FIG. 13A). To gain insight into specific details of receptor-ligand interactions, we employed ClusPro protein-protein docking to find pCTS-L/receptor complex configurations that exhibited the minimal Gibbs Free Energy (FIG. 13B). In the most stable pCTS-L/receptor complexes, the epitope sequence (P13: SEQ ID NO: 5 and SEQ ID NO: 6) for protective mAbs was sequestered into the hydrophobic crevices of TLR4 (FIG. 13B, Left Panels) but positioned sideways in close proximity to the V-domain of RAGE (FIG. 13B, Right Panels). The possibly different physical hindrance arisen from the engagement of P13-binding mAb20 might underlie its divergent inhibition of pCTS-L interaction with TLR4 (by 55-fold, FIG. 13A, Left Panels) and RAGE (by 10-fold, FIG. 13A, Right Panels). It suggests that protective anti-pCTS-L mAbs attenuate pCTS-L-induced inflammation possibly by inhibiting its interaction with these putative PRR receptors.

### Anti-pCTS-L mAb Attenuated Collagen Antibody-Induced Arthritis (CAIA)-Induced Arthritis in Mice

To explore the therapeutic efficacy of anti-pCTS-L mAbs in rheumatoid arthritis, we employed an animal model of CAIA to induce arthritis in both male and female Balb/C mice. Mice were injected with a cocktail of five anti-collagen mAbs (α-CII) on day 0, and subsequently challenged with sublethal dose of endotoxin (LPS) three days later (FIG. 14A). At day 6 post initial anti-collagen (α-CII) challenge, mice with similar scores of redness and swelling were randomly assigned to either control vehicle or anti-pCTS-L mAb treatment group, so that the average scores of arthritis were rather similar between two experimental groups before the onset of anti-pCTS-L mAb (2H8A2, 2.0 mg/kg) therapy (daily for 5 consecutive days: FIG. 14A). Repetitive administration of an anti-pCTS-L mAb significantly attenuated the progression of CAIA-induced arthritis (FIG. 14B), supporting a promising potential of pCTS-L-neutralizing mAbs in the treatment of human rheumatoid arthritis.

Because our monoclonal antibodies are produced in mice, they will likely trigger harmful immune responses in humans because human immune systems normally recognize murine antibodies as foreign proteins. These harmful immune responses often lead to rapid clearance of these foreign antibodies from the bloodstream, thereby rendering them ineffective. To circumvent this limit, one can use “humanized” antibodies that closely resemble human antibodies to reduce the likelihood of harmful immune responses to foreign proteins (52). In addition, some regulatory agencies, such as the U.S. Food and Drug Administration (FDA), often require that therapeutic antibodies intended for human use be humanized to ensure safety and efficacy. In order to humanize therapeutic antibodies for human sepsis and arthritis, the murine-derived monoclonal antibodies are modified by replacing their non-human framework (FR) regions with human counterparts while preserving the antibody's Complementarity-determining regions (CDRs) to maintain their ability to bind to the target antigen. Humanized antibodies offer both therapeutic effectiveness and compatibility with the human immune system, making them suitable for human clinical use.

In summary, we demonstrated that pCTS-L was induced by an intermediate inflammatory mediator to contribute to its systemic accumulation in experimental and clinical sepsis. Extracellular pCTS-L then interacted with both TLR4 and RAGE to induce dysregulated inflammation, Casp-11 activation, DAMP release, and tissue injury (FIG. 15). It now appears that pCTS-L use both TLR4 and RAGE to trigger dysregulated inflammation as manifested by the induction of various cytokines (e.g., TNF and IL-6) and chemokines (e.g., MCP-1, IL-8, MCP-1, GRO-ax/KC. GRO-R/MIP-2) in innate immune cells (FIG. 15). Likewise, pCTS-L might also trigger dysregulated immunosuppression partly by activating Casp-11, pyroptosis (FIG. 15), and passive release of pathogenic DAMPs (such as HMGB1 and SQSTM1). Furthermore, we have developed a panel of neutralizing mAbs capable of recognizing both murine and human pCTS-L to impair their interaction with both TLR4 and RAGE receptors, thereby rescuing mice from lethal sepsis even when the first dose was given in a delayed fashion. Furthermore, pCTS-L-neutralizing mAbs similarly attenuated CAIA-induced arthritis in mice, supporting therapeutic potential of pCTS-L-inhibiting mAbs in the treatment of human sepsis, rheumatoid arthritis, as well as other inflammatory diseases.

### Methods

### Study Design

All experiments were designed and performed in a rigorous fashion according to the principles and fundamentals of Good Laboratory Practice, with extensive attention to appropriate controls, statistically powered number of observations, as well as adequate number of replications to ensure reproducibility. The aim of this study was to assess the pathogenic changes of plasma pCTS-L concentrations in critically ill patients with sepsis, and to develop pCTS-L domain-specific monoclonal antibodies to prevent septic injury and lethality in pre-clinical settings. For the clinical investigation, blood samples were obtained from normal healthy controls and patients with sepsis or septic shock recruited to the Northwell Health System, and their plasma pCTS-L concentrations were assessed by immunoassays. To provide a cohort of age-matched normal healthy controls, we also obtained several healthy control serum samples from the Discovery Life Science Open Access Biorepository. Sample sizes were purely based on availability, and no blinding or randomization was applied for these non-interventional observations. For the pre-clinical study, animals were randomly assigned to different experimental groups, and treated with recombinant pCTS-L or pCTS-L-targeting antibodies at the indicated dosing regiments. The outcomes included animal survival rates and tissue histology scores, which were collected under blinded experimental conditions, so the identity of the groups was not revealed until after the completion of the experiments. Study design and sample sizes used for each experiment were provided in the figure legends. No data, including outlier values, were excluded.

### Cell Culture

Murine macrophage-like RAW 264.7 cells were obtained from ATCC. Primary peritoneal macrophages were isolated from wild-type Balb/C, wild-type C57BL/6, or mutant C57BL/6 mice defective either in TLR4 or both TLR4 and RAGE (7-8 wk, 20-25 g, male or female) at 3 days after intraperitoneal injection of 2 ml thioglycolate broth (4%) as previously described (23, 24, 53). Human blood was purchased from the New York Blood Center (Long Island City, NY. USA), and human PBMCs were isolated by density gradient centrifugation through Ficoll (Ficoll-Paque PLUS) as previously described (23, 24, 53). Murine macrophages and human PBMCs were cultured in DMEM supplemented with 1% penicillin/streptomycin and 10% FBS or 10% human serum. When they reached 70-80% confluence, adherent cells were gently washed with, and immediately cultured in, OPTI-MEM I before stimulating with crude LPS (E. coli 0111:B4, #L4130. Sigma-Aldrich), recombinant human SAA (Cat. #300-13, PeproTech), HMGB1, or pCTS-L. The intracellular and extracellular concentrations of pCTS-L or various other cytokines/chemokines were determined by Western blotting analysis, Cytokine Antibody Arrays or ELISA as previously described (23, 24, 53).

### MALDI-TOF Mass Spectrometry

To identify the 40-kDa band that was induced by human SAA in macrophage-conditioned culture medium, proteins in the cell-conditioned culture medium were resolved by SDS-PAGE gel electrophoresis, and the corresponding 40-kDa band was subjected to MALDI-TOF mass spectrometry analysis as previously described (26, 53). Briefly, the 40-kDa band was excised from the SDS-PAGE gel, and subjected to in-gel trypsin digestion. The mass of the tryptic peptides was measured by MALDI-TOF-MS and then subjected to peptide mass fingerprinting database analysis to identify the 40-kDa protein (“P40”).

### Cytokine Antibody Arrays

Murine Cytokine Antibody Arrays (Cat. No. AAM-CYT-3-8, RayBiotech Inc., Norcross, GA, USA), which simultaneously detect 62 cytokines on one membrane, were used to measure relative cytokine concentrations in macrophage-conditioned culture medium or murine serum as described previously (23, 53). Human Cytokine Antibody C3 Arrays (Cat. No. AAH-CYT-3-8), which detect 42 cytokines on one membrane, were used to determine cytokine concentrations in human PBMC-conditioned culture medium or human plasma samples as previously described (23, 24, 53).

### Open Surface Plasmon Resonance (SPR)

We employed the Nicoya Lifesciences gold-nanoparticle-based Open Surface Plasmon Resonance (OpenSPR) technology (Kitchener. ON, Canada) to characterize protein-protein interactions following the manufacturer's instructions. For instance, highly purified recombinant pCTS-L, human TLR4 (ECD-His Tag, residue 1-631, Cat. #10146-H08B, Sino Biological Inc) or extracellular domain of human RAGE (residue 1-344, Cat. #11629-H08H, Sino Biological Inc.) was respectively immobilized on NTA sensor chip (Cat. #SEN-Au-100-10-NTA), and TLR4, RAGE or pCTS-L was applied as analyte at different concentrations. To determine the binding affinities of mAbs to human or murine pCTS-L, highly purified human or murine pCTS-L was immobilized on the NTA sensor chip (Cat. #SEN-Au-100-10-NTA), and various mAbs were applied at various concentrations. The response units were recorded over time, and the binding affinity was estimated as the equilibrium dissociation constant KD using the Trace Drawer Kinetic Data Analysis v.1.6.1. (Nicoya Lifesciences).

### ClusPro Protein-Protein Docking

To gain insight into specific details of receptor-ligand interactions, we used the ClusPro Web Server (https://cluspro.org) (54) for protein-protein docking to find structures of the pCTS-L/receptor complexes that exhibited the minimal Gibbs Free Energy under the assumption that conformational changes were moderate upon protein-protein interactions.

Docking with each energy parameter set resulted in ten models defined by centers of highly populated clusters of lowest Gibbs Free Energy docked structures. We selected the model with the lowest Gibbs Free Energy to predict possible structure of the pCTS-L/receptor complexes.

### Animal Model of Lethal Endotoxemia and Sepsis

This study was conducted in accordance with policies of the NIH Guide for the Care and Use of Laboratory Animals and approved by the IACUC of the FIMR. To evaluate the role of pCTS-L in lethal sepsis. Balb/C mice (male or female, 7-8 weeks old, 20-25 g) were subjected to lethal endotoxemia or sepsis by intraperitoneal administration of bacterial endotoxins (LPS, E. coli 0111:B4, #L4130, Sigma-Aldrich) or by a surgical procedure termed as cecal ligation and puncture (CLP) as previously described (23, 24, 53). Briefly, the cecum of Balb/C mice was ligated at 5.0 mm from the cecal tip, and then punctured once with a 22-gauge needle. To alleviate surgical pain, all animals were given a dose of anesthetics (e.g., buprenorphine, 0.05 mg/kg, s.c.) immediately prior to CLP surgery, and small amounts of anesthetics (such as bupivacaine and lidocaine) locally around the incision site immediately after the closure of the abdominal wound of CLP surgery. At 30 min after CLP, all animals were given a subcutaneous dose of imipenem/cilastatin (0.5 mg/mouse) (Primaxin, Merck & Co., Inc.) and resuscitation with normal sterile saline solution (20 ml/kg). Recombinant pCTS-L or anti-pCTS-L polyclonal or monoclonal IgGs were intraperitoneally administered to endotoxemic or septic mice at the indicated doses and time points, and animal survival rates were monitored for up to two weeks.

### Real Time RT-PCR Analysis of Ctsl Expression.

Male Balb/C mice were subjected to lethal sepsis by cecal ligation and puncture (CLP), and animals were sacrificed at 24 h post CLP. Total RNAs were isolated from various tissues using the Trizol reagent kit, and reversely transcribed into the first-strand cDNA. Following reverse transcription, quantitative PCR was performed with Taqman Master Mix (ThermoFisher Scientific) for murine Ctsb (#4351370, Assay ID Mm01310506 m1), Ctse (#4351370, Assay ID Mm00456010_m1), Ctsl (#4351370, Assay ID Mm00515597 m1), Ctss (#4351370, Assay ID Mm01255859_m1), and glyceraldehyde 3-phosphate dehydrogenase gene (#4351370, Assay ID Mm99999915-g1) to quantify the mRNA expression levels of respective genes using a ABI 7900HT Fast Real-time PCR system. The relative abundance of various Cts mRNA expression in control group was normalized by GAPDH.

### Genotyping

We obtained a few breeding pairs of heterozygous Ctsl-KO (NOD.129P2(B6)-Ctsl^{tmCptr}/Rcl J) mice from the Jackson Laboratory (Stock No. 008352, Bar Harbor, ME. USA), and bred heterozygous Ctsl^{+/-} females with heterozygous Ctsl^{+/-} males to produce homozygous Ctsl KO as well as wild-type littermates for the proposed animal studies. To verify the genotypes of wild-type (WT) and Ctsl KO mice, tail biopsies was digested in Direct-PCR lysis Reagent (Cat. No 102-T, Viagen Biotech, Inc.) containing 0.4 μg/ml proteinase K (Cat. No E00491. ThermoFisher Scientific), and lysate containing genomic DNA was amplified by PCR reaction using the following primers: forward primer for WT Cts: 5′-GGA GGA GAG CGA TAT GGG-3′(SEQ ID NO: 28); reverse primer for WT Ctsl: 5′-AGC CAT TCA CCA CCT GCC-3 SEQ ID NO: 29); forward primer for mutant Ctsl: 5′-AAT TCG CCA ATG ACA AGA CG-3′ (SEQ ID NO: 30), under the following conditions: 95 °C 3 min; followed by 37 cycles of 95 °C for 15 sec and 60 °C for 15 sec, 72 °C, 15 sec. The PCR products were resolved on a 2% agarose gel and visualized by ethidium bromide staining. We subjected the genetic background-, age-, and sex-matched wild-type littermate (Ctsl^{+/+}) and pCTS-L-deficient (Ctsl^{-/-}) mice to lethal sepsis, and compared sepsis-induced liver injuries between wild-type and Ctsl-deficient mice at 24 h post CLP.

### Tissue Injury

The liver and intestine samples were harvested at 24 h post CLP or pCTS-L intraperitoneal administration, and fixed in 10% buffered formalin before being embedded in paraffin. Paraffin-embedded tissues were cut into 5-μm sections, stained with hematoxylin-eosin and examined under light microscopy. As previously described (55), liver parenchymal injury was assessed in a blinded fashion by the sum of three different Suzuki scores ranging from 0-4 for sinusoidal congestion, vacuolization of hepatocyte cytoplasm, and parenchymal necrosis. Using a weighted equation with a maximum score of 100 per field, the parameter scores were calculated and then averaged as the final liver injury score in each experimental group.

### RNA-Seq Analysis

To elucidate the mechanisms underlying pCTS-L-mediated pathogenesis, normal healthy Balb/C mice were intraperitoneally given recombinant pCTS-L (20 mg/kg), and various tissues were harvested 24 h later to isolate total RNA to characterize the expression profile of a full catalog of transcripts by RNA Sequencing (RNA-Seq, GENEWIZ, South Plainfield, NJ, USA). Gene ontology (GO) analysis and Kyoto Encyclopedia of Genes and Genomes (KEGG) pathway analysis were applied to analyze the differentially expressed genes (DEGs) by using String online tools (https string-db.org/cgi/input.pl). Differential expression analysis was performed using the Wald test (DESeq2) to generate P-values and log 2 fold changes. Genes with an adjusted P-value<0.05 and absolute log 2 fold change>2 were defined as differentially expressed.

### Western Blotting

The concentrations of pCTS-L in murine macrophage- or human PBMC-conditioned culture medium, murine serum or human plasma were determined by Western blotting analysis using commercial mAb (#C0994, Sigma-Aldrich) or pAb (Cat. #SC6498. Santa Cruz), or home-made rabbit anti-murine pCTS-L pAb. The concentrations of cellular pro-Casp-11 and mature Casp-11 in pCTS-L-stimulated peritoneal macrophages were determined by Western blotting using rabbit anti-mouse caspase-11 monoclonal antibodies (Cat. #ab180673, Abcam). Equal volume of cell-conditioned culture medium or murine/human serum were resolved on sodium dodecyl sulfate (SDS)-polyacrylamide gels and transferred to polyvinylidene difluoride (PVDF) membranes. After blocking with 5% nonfat milk, the membranes were incubated with the appropriate antibodies (anti-pCTS-L, 1:1000; anti-pro-Casp-11, 1:1000) overnight. Subsequently, the membranes were incubated with the appropriate secondary antibodies, and the immune-reactive bands were visualized by chemiluminescence. The relative levels of specific proteins were determined using the UN-SCAN-IT Gel Analysis Software Version 7.1 (Silk Scientific Inc., Orem, UT, USA) with reference to appropriate controls. The liver content of fibrinogen-γ (FGG) was measured by Western blotting analysis using mouse anti-FGG monoclonal antibody following standard procedures with reference to a house-keeping protein, β-actin.

Wild-type Ctsl^{+/+} or Ctsl^{-/-} KO (NOD.129P2(B6)-Ctsl^{tmCPtr}/Rcl J) mice were intraperitoneally administered with bacterial endotoxin (8.0 mg/kg), and animals were sacrificed at 24 h post endotoxemia to harvest blood. The concentration of pCTS-L in murine serum was determined by Western blotting analysis using home-made rabbit anti-murine pCTS-L polyclonal antibodies. Equal volume of serum was resolved on sodium dodecyl sulfate (SDS)-polyacrylamide gels and transferred to polyvinylidene difluoride (PVDF) membranes. After blocking with 5% nonfat milk, the membranes were incubated with the appropriate antibodies (anti-pCTS-L, 1:1000) overnight. Subsequently, the membranes were incubated with the appropriate secondary antibodies, and the immune-reactive bands were visualized by chemiluminescence.

### ELISA

To confirm the relative cytokines levels, ELISA kits were used to quantitate the concentrations of pCTS-L in parallel with several biomarkers of experimental and clinical sepsis. An ELISA kit for human pCTS-L (Cat. #MBS7254442, MyBioSource.com) was used measure blood pCTS-L levels in normal healthy controls and septic patients. In addition, we obtain ELISA kits for other inflammatory biomarkers including GRO (Cat. #ELH-GRO-1, RayBiotech), IL-6 (Cat. #MBS8123859, MyBioSource), IL-8 (Cat. #ELH-IL8, RayBiotech), MCP-1 (Cat. #MBS7721397), and HMGB1 (Cat. #OKCD03560, Aviva Systems Biology) to measure their levels in septic patients as well as in gender-matched normal healthy controls.

To measure extracellular SQSTM1 in macrophage-conditioned culture medium, an ELISA kit was obtained from the Novus Biological Inc. (Cat. #NBP2-61300). Peritoneal macrophages were isolated from wild-type C57BL/6 mice (male, 8-10 Weeks, 20-25 g) or mutant mice deficient in both TLR4 and RAGE at 3d post intraperitoneal administration of thioglycolate broth, and stimulated with recombinant murine pCTS-L for 6 h or 24 h in serum-free DMEM medium. Macrophage-conditioned culture medium were collected, and extracellular levels of KC and MIP-2 were measured by using mouse KC/CXCL1 Douset ELISA Kit (Cat. #DY453-05, R&D System) and mouse MIP-2/CXCL2 Q Douset ELISA Kit (Cat. #DY452-05. R&D Systems).

Preparation of Recombinant Human and Murine pCTS-L Proteins

The cDNA encoding for human (residue 17-333) and murine (residue 18-334) pCTS-L was cloned into a pReceiver expression vector downstream of a T7 promoter with an N-histidine tag, and recombinant pCTS-L protein was expressed in E. coli BL21 (DE3) pLysS as previously described (53). The inclusion body-associated recombinant pCTS-L protein was isolated by differential centrifugation and urea solubilization before refolding in Tris buffer (pH 8.0) containing N-lauroylsarcosine. The recombinant pCTS-L protein with N-His Tag was then further purified by histidine-affinity chromatography, followed by extensive Triton X-114 extractions to remove contaminating endotoxins. Recombinant pCTS-L protein was tested for LPS content by the chromogenic Limulus amebocyte lysate assay (Endochrome; Charles River), and the endotoxin content was less than 0.01 U per microgram of recombinant protein. For comparison, we also obtained bacterial products-free pCTS-L expressed in human HEK293 cells (Cat. #. CTT-H5222, Acro Biosystems) as an additional control for recombinant protein expressed in E. coli.

### Generation of Anti-pCTS-L Polyclonal and Monoclonal Antibodies

Polyclonal antibodies were generated in Female New Zealand White Rabbits by the Covance Inc. (Princeton, NJ, USA) using recombinant murine and human pCTS-L in combination with Freund's complete adjuvant following standard procedures. Blood samples were collected in 3-week cycles of immunization and bleeding, and the antibody titers were determined by direct pCTS-L ELISA. Total IgGs and pCTS-L antigen-binding IgGs were purified from anti-pCTS-L rabbit serum using Protein A and pCTS-L-affinity chromatography as described in the Supplemental Materials. The monoclonal antibodies were generated in Balb/C and C57BL/6 mice by the GenScript (Piscataway, NJ, USA) using highly purified human or murine pCTS-L following standard procedures. Blood samples were collected every two weeks, and serum titers were assessed by indirect ELISA and Western blotting analysis. After four immunizations, mouse splenocytes were harvested, fused with mouse Sp2/0 myeloma cell line, and screened for antibody-producing hybridomas by indirect ELISA, dot blotting, and Western blotting analysis. After limiting dilution, purified hybridoma clones were generated to produce mAbs following standard procedures.

### Affinity Purification of Polyclonal Antibodies

Total IgGs and pCTS-L antigen-binding IgGs were purified from anti-pCTS-L rabbit serum using Protein A and pCTS-L-affinity column chromatography, respectively. Briefly, rabbit serum was pre-buffered with PBS and slowly loaded onto the Protein A/G Sepharose (Cat. #ab193262) column to allow sufficient binding of IgGs. After washing with 1×PBS to remove unbound serum components, the IgGs were eluted with acidic buffer (0.1 M glycine-HCl, pH 2.8), and then immediately dialyzed into 1×PBS buffer at 4 °C overnight. For pCTS-L antigen-affinity purification, recombinant murine pCTS-L was conjugated to cyanogen bromide (CNBr)-activated Sepharose4 agarose beads (Cat. #17098101, GE Healthcare), and the pCTS-L-conjugated Sepharose beads were then loaded onto columns. Following repetitive washings with acid buffer (0.1 M Acetic/Sodium Acetate, 0.5 M NaCl, pH4.0) and alkali buffer (0.1 M Tris-HCl, 0.5 NaCl, pH 8.0), anti-pCTS-L total IgGs were slowly loaded onto the column, and the flow-through fractions were collected. Following repetitive washing with 1×PBS buffer, the pCTS-L-binding antibodies were eluted with acidic elution buffer, and immediately neutralized in 1×PBS buffer.

### Peptide Dot Blotting

A library of 24 synthetic peptides corresponding to different regions of human or murine pCTS-L sequence were synthesized at the Genscript, and spotted (0.1 μg in 2.5 μl) onto nitrocellulose membrane (Thermo Scientific, Cat No. 88013). Subsequently, the membrane was probed with anti-pCTS-L rabbit or murine serum, or IgGs isolated from anti-pCTS-L rabbit serum or murine hybridoma cultures following a standard protocol.

### Clinical Characterization of Septic Patients

This study was approved by the institutional review board (IRB) of the Feinstein Institutes for Medical Research (FIMR) and endorsed by written informed consent from all participants providing blood samples. Blood samples (5.0 ml) were collected from eight healthy control subjects and ten septic patients recruited to North Shore University Hospital and the Long Island Jewish Medical Center between 2018-2019 (listed as “P1-P10” in the data file S1). Patients were diagnosed with sepsis or septic shock by the Sepsis-3 criteria (56), and blood samples (5.0 ml) were obtained within 24 h of diagnosis of sepsis (defined as “time 0”), followed by two subsequent blood sampling at 24 and 72 h post the initial diagnosis. To provide a cohort of age-matched normal healthy controls, we also purchased several healthy control serum samples from the Discovery Life Science Open Access Biorepository. Subsequently, these clinical samples were assayed for pCTS-L by Western blotting and human pCTS-L ELISA kit (Cat. #MBS7254442. MyBioSource.com) with reference to purified recombinant human pCTS-L at various dilutions.

### Animal Model of Collagen-Antibody-Induced Arthritis

Collagen antibody-induced arthritis (CAIA) was induced in Balb/C mice (14-16 weeks, 21-28 g) by administering a cocktail of anti-collagen type II mAbs (α-CII, Cat. #53010, Chondrex, Woodinville, WA, USA; 1.4-1.6 mg/mouse) against conserved auto-antigenic epitopes of collagen type II (CII), followed by an additional endotoxin challenge (25-30 μg/mouse) 3 days later (57). The α-CII cocktail contains five different mAbs [A2-10 (IgG2a), D1-2G (IgG2b), D2-112 (IgG2b), F10-21 (IgG2a), and D8-6 (IgG2a)] recognizing conserved epitopes clustered within the 167 amino acid fragment called LyC1 (124-290) or the 83 amino acid peptide fragment called LyC2 (291-374) of the CB11 fragment (124-402). These anti-collagen antibodies trigger a robust inflammatory response against a specific protein present in the extracellular matrix of joint tissues (e.g., collagen), leading to synovial inflammation, cartilage degradation, and bone erosion that closely mimics the features of human rheumatoid arthritis (57). Endotoxin, however, induces systemic production of many cytokines/chemokines which further accelerate and improve the synchronization and severity of the disease (58). On day 6 post initial anti-collagen antibody challenge, mice were intraperitoneally administered with either 0.9% saline (as control vehicle) or a pCTS-L-neutralizing mAb daily for 5 consecutive days. Animals were carefully examined daily post LPS challenge and the severity of rheumatoid arthritis was scored (up to 4 points per joint, maximum=16 points per mouse) according the following criteria: “0”, no signs of redness (erythema) and swelling: “1”, erythema and mild swelling confined to one type of joint (the tarsals or ankle joint); “2”, erythema and mild swelling extending from the ankle to the tarsals thereby affecting two types of joint; “3”, erythema and moderate swelling extending from the ankle to metatarsal joints thereby affecting all three type of joint (entire paw): “4”, erythema and severe swelling encompass the ankle, foot and digits, or ankylosis of the limb thereby leading to the disappearance of anatomical definition.

### Statistical Analysis

All data were assessed for normality by the Shapiro-Wilk test before conducting appropriate statistical tests among groups. The comparison of two independent samples was assessed by the Student's t test and the Mann-Whitney test for Gaussian and non-Gaussian distributed samples, respectively. For comparison among multiple groups with normal data distribution, the differences were analyzed by one-way analyses of variance (ANOVA) followed by the Fisher Least Significant Difference (LSD) test. For comparison among multiple groups with non-normal (skewed) distribution, the statistical differences were evaluated with the non-parametric Kruskal-Wallis ANOVA test. For survival studies, the Kaplan-Meier method was used to compare the differences in mortality rates between groups with the nonparametric log-rank post hoc test. The Spearman rank or the Pearson correlation coefficient test was used to evaluate associations between two quantitative variables that exhibit non-normal or normal distribution, respectively. A P value<0.05 was considered statistically significant.

### Discussion

In this invention, we demonstrated that pCTS-L was induced by an intermediate inflammatory mediator and served as a late-acting mediator of lethal sepsis. This newly un-covered and novel role of extracellular pCTS-L in sepsis further expanded its pathogenic involvement in other inflammatory diseases such as pancreatitis (49), atherosclerosis (50), renal disease (59), vascular intimal hyperplasia (51), arthritis (41), and colitis (60). Inspired by our findings that recombinant pCTS-L worsened LPS-induced animal lethality, whereas pCTS-L-neutralizing polyclonal IgGs conferred a significant protection against sepsis, we employed the traditional hybridoma technology developed by Kohler and Milsteinin almost half century ago to generate a panel of potentially therapeutic mAbs. Specifically, we have developed a panel of neutralizing mAbs (2H8A2, 20D5H6 and 26C7C9) capable of recognizing both murine and human pCTS-L to impair their interaction with both TLR4 and RAGE receptors, thereby rescuing mice from lethal sepsis even when the first dose was given in a delayed fashion. Furthermore, these pCTS-L-neutralizing mAbs similarly attenuated the progression of rheumatoid arthritis in a murine model of anti-collagen antibody-induced arthritis.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### CITED PUBLICATIONS

1. K. E. Rudd et al., Global, regional, and national sepsis incidence and mortality, 1990-2017: analysis for the Global Burden of Disease Study. Lancet 395, 200-211 (2020).
2. E. W. Tindal, B. E. Armstead. S. F. Monaghan. D. S. Heffernan, A. Ayala, Emerging therapeutic targets for sepsis. Expert Opin Ther Targets 25, 175-189 (2021).
3. A. Poltorak et al., Defective LPS signaling in C3H/HeJ and C57BL/10ScCr mice: mutations in Tlr4 gene. Science 282, 2085-2088 (1998).
4. E. Hailman et al., Lipopolysaccharide (LPS)-binding protein accelerates the binding of LPS to CD14. J Exp Med 179, 269-277 (1994).
5. S. D. Wright, R. A. Ramos, P. S. Tobias, R. J. Ulevitch, J. C. Mathison, CD14, a receptor for complexes of lipopolysaccharide (LPS) and LPS binding protein. Science 249, 1431-1433 (1990).
6. K. J. Tracey et al., Anti-cachectin/TNF monoclonal antibodies prevent septic shock during lethal bacteraemia. Nature 330, 662-664 (1987).
7. C. A. Dinarello. Biologic basis for interleukin-1 in disease. Blood 87, 2095-2147 (1996).
8. F. P. Heinzel, The role of IFN-gamma in the pathology of experimental endotoxemia. J Immunol 145, 2920-2924 (1990).
9. S. K. Vanajaetal., Bacterial Outer Membrane Vesicles Mediate Cytosolic Localization of LPS and Caspase-11 Activation. Cell%19; 165, 1106-1119 (2016).
10. J. A. Hagar, D. A. Powell, Y. Aachoui, R. K. Ernst, E. A. Miao, Cytoplasmic LPS activates caspase-11: implications in TLR4-independent endotoxic shock. Science 341, 1250-1253 (2013).
11. H. Wang et al., HMG-1 as a late mediator of endotoxin lethality in mice. Science 285, 248-251 (1999).
12. B. Zhou et al., Extracellular SQSTM1 mediates bacterial septic death in mice through insulin receptor signalling. Nat. Microbiol 5, 1576-1587 (2020).
13. C. S. Zhu et al., Endogenous Regulation and Pharmacological Modulation of Sepsis-Induced HMGB1 Release and Action: An Updated Review. Cells 10, 2220 (2021).
14. H. Watanabe. M. Son, The Immune Tolerance Role of the HMGB1-RAGE Axis. Cells 10, 564 (2021).
15. J. Xu et al., Macrophage endocytosis of high-mobility group box 1 triggers pyroptosis. Cell Death. Differ 21, 1229-1239 (2014).
16. M. Deng et al., The Endotoxin Delivery Protein HMGB1 Mediates Caspase-11-Dependent Lethality in Sepsis. Immunity 49, 740-753 (2018).
17. M. Gregoire et al., Frontline Science: HMGB1 induces neutrophil dysfunction in experimental sepsis and in patients who survive septic shock. J. Leukoc. Biol 101, 1281-1287 (2017).
18. V. S. Patel et al., HMGB1 Mediates Hyperoxia-Induced Impairment of Pseudomonas aeruginosa Clearance and Inflammatory Lung Injury in Mice. Am. J. Respir. Cell Mol. Biol, (2012).
19. H. Yang et al., Reversing established sepsis with antagonists of endogenous high-mobility group box 1. Proc Natl Acad Sci USA 101, 296-301 (2004).
20. G. Ramadori, J. D. Sipe, C. A. Dinarello, S. B. Mizel, H. R. Colten, Pretranslational modulation of acute phase hepatic protein synthesis by murine recombinant interleukin 1 (IL-1) and purified human IL-1. J. Exp. Med 162, 930-942 (1985).
21. S. Sandri et al., Is serum amyloid A an endogenous TLR4 agonist? J. Leukoc. Biol 83, 1174-1180 (2008).
22. S. D. Yan et al., Receptor-dependent cell stress and amyloid accumulation in systemic amyloidosis [see comments]. Nat Med 6, 643-651 (2000).
23. W. Li et al., Connexin 43 Hemichannel as a Novel Mediator of Sterile and Infectious Inflammatory Diseases. Sci. Rep 8, 166-18452 (2018).
24. W. Chen et al., Enhanced Macrophage Pannexin 1 Expression and Hemichannel Activation Exacerbates Lethal Experimental Sepsis. Sci. Rep 9, 160-37232 (2019).
25. S. Zhu et al., High-Density Lipoprotein (HDL) Counter-Regulates Serum Amyloid A (SAA)-Induced sPLA2-IIE and sPLA2-V Expression in Macrophages. PLoS. One 11, e0167468 (2016).
26. W. Li et al., Serum Amyloid A Stimulates PKR Expression and HMGB1 Release Possibly through TLR4/RAGE Receptors. Mol. Med 21. 515-525 (2015).
27. D. J. Dowling et al., Major secretory antigens of the helminth Fasciola hepatica activate a suppressive dendritic cell phenotype that attenuates Th17 cells but fails to activate Th2 immune responses. Infect. Immun 78, 793-801 (2010).
28. K. Ishidoh, E. Kominami, Gene regulation and extracellular functions of procathepsin L. Biol. Chem 379, 131-135 (1998).
29. S. E. Kane, M. M. Gottesman, The role of cathepsin L in malignant transformation. Semin. Cancer Biol 1, 127-136 (1990).
30. E. Fiebiger et al., Invariant chain controls the activity of extracellular cathepsin L. J. Exp. Med 196, 1263-1269 (2002).
31. R. Lemaire et al., Selective induction of the secretion of cathepsins B and L by cytokines in synovial fibroblast-like cells. Br. J. Rheumatol 36, 735-743 (1997).
32. K. K. Kharbanda, D. L. McVicker, R. K. Zetterman, T. M. Donohue. Jr., Ethanol consumption alters trafficking of lysosomal enzymes and affects the processing of procathepsin L in rat liver. Biochim. Biophys. Acta 1291, 45-52 (1996).
33. H. Takahashi et al., Cathepsin L activity is increased in alveolar macrophages and bronchoalveolar lavage fluid of smokers. Am. Rev. Respir. Dis 147, 1562-1568 (1993).
34. A. Klose, A. Wilbrand-Hennes, J. Brinckmann, N. Hunzelmann, Alternate trafficking of cathepsin L in dermal fibroblasts induced by UVA radiation. Exp. Dermatol 19, e117-e123 (2010).
35. J. S. Smolen, D. Aletaha, I. B. McInnes. Rheumatoid arthritis. Lancet 388, 2023-2038 (2016).
36. I. B. McInnes. G. Schett. Pathogenetic insights from the treatment of rheumatoid arthritis. Lancet 389, 2328-2337 (2017).
37. C. S. Zhu et al., Identification of procathepsin L (pCTS-L)-neutralizing monoclonal antibodies to treat potentially lethal sepsis. Sci Adv 9, eadf4313 (2023).
38. Y. Iwata, J. S. Mort, H. Tateishi, E. R. Lee, Macrophage cathepsin L, a factor in the erosion of subchondral bone in rheumatoid arthritis. Arthritis Rheum 40, 499-509 (1997).
39. R. A. Maciewicz, S. F. Wotton, Degradation of cartilage matrix components by the cysteine proteinases, cathepsins B and L. Biomed Biochim Acta 50, 561-564 (1991).
40. T. Weitoft et al., Cathepsin S and cathepsin L in serum and synovial fluid in rheumatoid arthritis with and without autoantibodies. Rheumatology (Oxford) 54, 1923-1928 (2015).
41. U. Schurigt et al., Decreased arthritis severity in cathepsin L-deficient mice is attributed to an impaired T helper cell compartment. Inflamm. Res 61, 1021-1029 (2012).
42. C. Deval et al., Identification of cathepsin L as a differentially expressed message associated with skeletal muscle wasting. Biochem J 360, 143-150 (2001).
43. J. G. Heuer et al., Evaluation of protein C and other biomarkers as predictors of mortality in a rat cecal ligation and puncture model of sepsis. Crit Care Med 32, 1570-1578 (2004).
44. W. Li et al., A Major Ingredient of Green Tea Rescues Mice from Lethal Sepsis Partly by Inhibiting HMGB1. PLoS ONE 2, e1153 (2007).
45. F. A. Bozza et al., Cytokine profiles as markers of disease severity in sepsis: a multiplex analysis. Crit Care 11, R49 (2007).
46. K. R. Machlus, J. C. Cardenas, F. C. Church, A. S. Wolberg. Causal relationship between hyperfibrinogenemia, thrombosis, and resistance to thrombolysis in mice. Blood 117, 4953-4963 (2011).
47. R. L. Robker et al., Progesterone-regulated genes in the ovulation process: ADAMTS-1 and cathepsin L proteases. Proc. Natl. Acad. Sci. USA 97, 4689-4694 (2000).
48. W. W. Wright, L. Smith, C. Kerr, M. Charron, Mice that express enzymatically inactive cathepsin L exhibit abnormal spermatogenesis. Biol Reprod 68, 680-687 (2003).
49. T. Wartmann et al., Cathepsin L inactivates human trypsinogen, whereas cathepsin L-deletion reduces the severity of pancreatitis in mice. Gastroenterology 138, 726-737 (2010).
50. S. Kitamoto et al., Cathepsin L deficiency reduces diet-induced atherosclerosis in low-density lipoprotein receptor-knockout mice. Circulation 115, 2065-2075 (2007).
51. J. Cai et al., Cathepsin L promotes Vascular Intimal Hvperplasia after Arterial Injury. Mol Med 23, 92-100 (2017).
52. F. A. Harding. M. M. Stickler. J. Razo. R. B. DuBridge, The immunogenicity of humanized and fully human antibodies: residual immunogenicity resides in the CDR regions. MAbs 2, 256-265 (2010).
53. W. Chen et al., Identification of tetranectin-targeting monoclonal antibodies to treat potentially lethal sepsis. Sci. Transl. Med 12, 12/539 (2020).
54. D. Kozakov et al., The ClusPro web server for protein-protein docking. Nat Protoc 12, 255-278 (2017).
55. X. Qiang et al., Human Dermcidin Protects Mice Against Hepatic Ischemia-Reperfusion-Induced Local and Remote Inflammatory Injury. Front Immunol 12, 821154 (2021).
56. M. Shankar-Hari et al., Developing a New Definition and Assessing New Clinical Criteria for Septic Shock: For the Third International Consensus Definitions for Sepsis and Septic Shock (Sepsis-3). JAMA 315, 775-787 (2016).
57. L. M. Khachigian, Collagen antibody-induced arthritis. Nat Protoc 1, 2512-2516 (2006).
58. A. R. Moore et al., Collagen II antibody-induced arthritis in Tg1278TNFα mice: optimization of a novel model to assess treatments targeting human TNFα in rheumatoid arthritis. J Transl Med 12, 285 (2014).
59. Y. Cao et al., Cathepsin L activity correlates with proteinuria in chronic kidney disease in humans. Int. Urol. Nephrol 49, 1409-1417 (2017).
60. C. Bauer et al., Colitis induced in mice with dextran sulfate sodium (DSS) is mediated by the NLRP3 inflammasome. Gut 59, 1192-1199 (2010).

1. A monoclonal antibody specifically binding to amino acid sequence GGLDSEESYPYEATEESCKYN (SEQ ID NO: 6) present in residues 194-214 of human precathepsin L (SEQ ID NO: 2), or an antigen-binding fragment thereof, comprising a heavy chain variable region (VH) and a light chain variable region (VL), wherein
(A)
(i) the heavy chain variable region (VH) comprises:
(a) an amino acid sequence of heavy chain CDR1 comprising the amino acid sequence DTYMH (SEQ ID NO: 31);
(b) an amino acid sequence of heavy chain CDR2 comprising the amino acid sequence RIDPAIVNTRYDPKFQD (SEQ ID NO: 32); and
(c) an amino acid sequence of heavy chain CDR3 comprising the amino acid sequence LGNYYGSRYVMDY (SEQ ID NO: 33); and
(ii) the light chain variable region (VL) comprises:
(a) an amino acid sequence of light chain CDR1 comprising the amino acid sequence RSSQSLVHSNGNTYLH (SEQ ID NO: 34);
(b) an amino acid sequence of light chain CDR2 comprising the amino acid sequence KVSNRFS (SEQ ID NO: 35); and
(c) an amino acid sequence of light chain CDR3 comprising the amino acid sequence SQSTHVPYT (SEQ ID NO: 36);
or
(B)
(i) the heavy chain variable region (VH) comprises:
(a) an amino acid sequence of heavy chain CDR1 comprising the amino acid sequence NTYMH (SEQ ID NO: 37);
(b) an amino acid sequence of heavy chain CDR2 comprising the amino acid sequence RIDPTNGNTKYAPKFQG (SEQ ID NO: 38); and
(c) an amino acid sequence of heavy chain CDR3 comprising the amino acid sequence GDYGDGAY (SEQ ID NO: 39); and
(ii) the light chain variable region (VL) comprises:
(a) an amino acid sequence of light chain CDR1 comprising the amino acid sequence RSSQSLVHSNGNTYLH (SEQ ID NO: 34);
(b) an amino acid sequence of light chain CDR2 comprising the amino acid sequence KVSNRFS (SEQ ID NO: 35); and
(c) an amino acid sequence of light chain CDR3 comprising the amino acid sequence SQSTHVPYT (SEQ ID NO: 36);
or
(C)
(i) the heavy chain variable region (VH) comprises:
(a) an amino acid sequence of heavy chain CDR1 comprising the amino acid sequence NTYMY (SEQ ID NO: 40);
(b) an amino acid sequence of heavy chain CDR2 comprising the amino acid sequence RIDPANGNIKYDPKFQG (SEQ ID NO: 41); and
(c) an amino acid sequence of heavy chain CDR3 comprising the amino acid sequence RKVMTADYFDY (SEQ ID NO: 42); and
(ii) the light chain variable region (VL) comprises:
(a) an amino acid sequence of light chain CDR1 comprising the amino acid sequence RSSQSLVHSNGNTYLH (SEQ ID NO: 34);
(b) an amino acid sequence of light chain CDR2 comprising the amino acid sequence KVSNRFS (SEQ ID NO: 35); and
(c) an amino acid sequence of light chain CDR3 comprising the amino acid sequence SQSTHVPWT (SEQ ID NO: 43).

2. The antibody of claim 1, wherein the antibody is a humanized antibody.

3. A monoclonal antibody that specifically binds to amino acid sequence GGLDSEESYPYEATEESCKYN (SEQ ID NO: 6), wherein the monoclonal antibody has at least 90% sequence identify to the antibody according to claim 1.

4. The antibody or antigen-binding fragment thereof according to claim 1, wherein
(i) the heavy chain variable region (VH) comprises the amino acid sequences of SEQ ID NOs: 8, 12 and 16 as amino acid sequences of FR1, FR2, FR3 and FR4, respectively; and
(ii) the light chain variable region (VL) comprises the amino acid sequences of SEQ ID NOs: 10, 14, and 18 as amino acid sequences of FR1, FR2, FR3 and FR4, respectively.

5. The antibody or antigen-binding fragment thereof according to claim 1, wherein
(i) the heavy chain variable region (VH) comprises an amino acid sequence derived from the mouse mAb2, mAb20, and mAb26 H chain, respectively, and
(ii) the light chain variable region (VL) comprises an amino acid sequence derived from the mouse antibody mAb2, mAb20, and mAb26 L chain, respectively.

6. The antibody or antigen-binding fragment thereof according to claim 1, wherein
(i) the heavy chain variable region (VH) comprises an amino acid sequence having a 90% or more identity with the amino acid sequence of SEQ ID NO: 8, 12, and 16, respectively and
(ii) the light chain variable region (VL) comprises an amino acid sequence having a 90% or more identity with the amino acid sequence of SEQ ID NO: 10, 14, and 18, respectively.

7. The antibody or antigen-binding fragment thereof according to claim 1, wherein the antibody is a humanized antibody and the class (subclass) of the humanized antibody is IgG1(λ) or IgG2(λ).

8. The antibody or antigen-binding fragment thereof according to claim 1, wherein the binding activity thereof to human pCTS-L protein (analyzed by OpenSPR) is 10-fold lower than that of murine pCTS-L.

9. A pharmaceutical composition comprising the humanized antibody or antigen-binding fragment thereof according to claim 1 and a pharmaceutically acceptable carrier.

10. The pharmaceutical composition according to claim 9 for use in treatment or prevention of various diseases where pCTS-L is pathologically overexpressed, such as sepsis, rheumatoid arthritis, and other inflammatory diseases such as COVID-19.

11. An isolated nucleic acid encoding the amino acid sequence of the antibody or antigen-binding fragment thereof of claim 1, as described in SEQ ID NOs 7, 9, 11, 13, 15, or 17, or an isolated nucleic acid hybridizable with any of these nucleic acids under high stringent conditions.

12. A recombinant expression vector comprising the isolated nucleic acid according to claim 11.

13. A host cell transformed with the recombinant expression vector according to claim 12.

14. A method of treating sepsis, rheumatoid arthritis or an inflammatory disease in a patient in need thereof comprising administrating to the patient a therapeutically effective amount of the antibody or antigen-binding fragment thereof according to claim 1.

15. The method of claim 14, wherein the inflammatory disease is one or more of pancreatitis, atherosclerosis, chronic kidney disease, end-of stage renal disease, vascular injury, antigen-induced arthritis, dextran sulfate sodium (DSSD)-induced colitis and Covid-19.

16. The method of claim 14, wherein the inflammatory disease is one or more of Inflammatory conditions treated or attenuated by reducing circulating cytokine levels in a subject in need thereof include appendicitis, peptic, gastric or duodenal ulcers, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, Alzheimer's disease, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, ankylosing spondylitis, Type I diabetes, ankylosing spondylitis, Berger's disease, reactive arthritis (Reiter's syndrome) or Hodgkin's disease. In more preferred embodiments, the condition is appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection and graft-versus-host disease.
